(19) United States
(12) Patent Application Publication
(10) Pub. No.: US 2023/0271928 A1
(43) Pub. Date: Aug. 31, 2023

(54) NAPHTHYLUREA COMPOUND, METHODS OF PREPARATION AND USE THEREOF

(71) Applicant: Henan Radiomedical Science and Technology Co., Ltd., Kaifeng (CN)

(72) Inventors: Marvin Xuejun XU, Kaifeng (CN); Yupo YANG, Kaifeng (CN); Zhengyan YANG, Kaifeng (CN); Hongyun XU, Kaifeng (CN); Chaoqun DUAN, Kaifeng (CN); Zun ZHANG, Kaifeng (CN); Shaohua ZHANG, Kaifeng (CN)

(21) Appl. No.: 18/313,365

(22) Filed: May 7, 2023

Related U.S. Application Data
(63) Continuation of application No. PCT/CN2022/077027, filed on Feb. 21, 2022.

(30) Foreign Application Priority Data
Feb. 6, 2021 (CN) 202110165298.1

(51) Int. Cl. C07D 295/155

(52) U.S. Cl. CPC C07D 295/155

(57) ABSTRACT

The disclosure provides a naphthylurea compound having a formula I. R represents H, a C1-C5 straight-chain alkyl, a C1-C5 straight-chain alkyl with a halogen-substituted end or a 5-8-membered cycloalkyl; R₁, R₂, R₃, R₄, R₅, R₆, R₇, R₈, R₉ at each occurrence represent H, F, Cl, Br, —CN, —CH₃, —CF₃, —OCH₃, or —OCF₃; R₅ optionally represents phenyl, and M is H or —CH₃; m represents a number of CH₂, and is 0 or 1; n represents a number of CH₂, and is 1, 2, 3, or 4; and p represents a number of CH₂, and is 2; and X is O or S.

### CROSS-REFERENCE TO RELATED APPLICATIONS

This application is a continuation-in-part of International Patent Application No. PCT/CN2022/077027 with an international filing date of Feb. 21, 2022, designating the United States, now pending, and further claims foreign priority benefits to Chinese Patent Application No. 202110165298.1 filed Feb. 6, 2021. The contents of all of the aforementioned applications, including any intervening amendments thereto, are incorporated herein by reference. Inquiries from the public to applicants or assignees concerning this document or the related applications should be directed to: Matthias Scholl P. C., Attn.: Dr. Matthias Scholl Esq., 245 First Street, 18th Floor, Cambridge, Mass. 02142.

### INCORPORATION-BY-REFERENCE OF SEQUENCE LISTING

This application contains a sequence listing, which has been submitted electronically in XML file and is incorporated herein by reference in its entirety. The XML file, created on Mar. 6, 2023, is named ZZLK-03201-UUS.xml, and is 8,132 bytes in size.

### BACKGROUND

The disclosure relates to the field of target therapy for cancer, and more particularly, to a naphthylurea compound, methods of preparation and use thereof.

Conventional drugs for chemotherapy such as paclitaxel, cisplatin, and doxorubicin effectively inhibit the growth of cancer in the early stage. When the cancer cells are drug resistant, the drugs are ineffective. Novel targeted anticancer drugs such as trastuzumab, gefitinib, and solatinib still leave much to be desired in efficacy of chemotherapy.

A typical cell cycle contains four distinct phases that progress in an orderly fashion. The four distinct phases of the cell cycle are G1 (G for gap), S (Synthesis), G2, and M (Mitosis). Each phase of the cell cycle is monitored by internal controls called checkpoints. Cellular responses to stresses such as oxygen free radicals, ultraviolet radiation, chemical drugs and heavy metals are often accompanied by cell cycle arrest, which provides a temporal delay necessary to repair cell damage. A G1/S checkpoint and a G2/M checkpoint are initiated in response to major events of the cell cycle, such as DNA replication, protein synthesis and cell division, thereby maintaining the structure and function of a genome.

Conventional cancer treatment utilizes radiotherapy and chemotherapy drugs to induce genomic instability, resulting in apoptosis of the cancer cells. The conventional cancer treatment also induces cell cycle arrest in response to DNA damage, so that the cancer cells become resistant to therapeutic drugs. The cell cycle checkpoints may fail because genes or proteins are commonly mutated in malignant tumors. If the G1/S checkpoint fails, tumor cells mainly rely on the G2/M checkpoint to halt the cell cycle in order to repair DNA damage. Therefore, an important tumor suppression strategy is to selectively disrupt the cell cycle checkpoints, thus enhancing the sensitivity of tumors to damage.

### SUMMARY

The disclosure provides a naphthylurea compound, uses of derivatives thereof in treatment of tumor, targets thereof, and an anti-tumor mechanism thereof. By some biological analysis techniques, the naphthylurea compound has been found to be effective anti-tumor agents that inhibit proliferation and development of tumor cells within liver cancer, breast cancer, lung cancer and leukemia, causing the tumor cells to be arrested in the G2/M phase of the cell cycle and undergo apoptosis.

The objective of the disclosure is to provide a naphthylurea compound, methods of preparation and use thereof.

The naphthylurea compound have the following formula I:

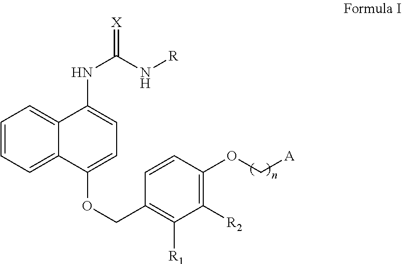

where R represents H, a C₁-C₅ straight-chain alkyl, a C₁-C₅ straight-chain alkyl with a halogen-substituted end, a 5-8-membered cycloalkyl,

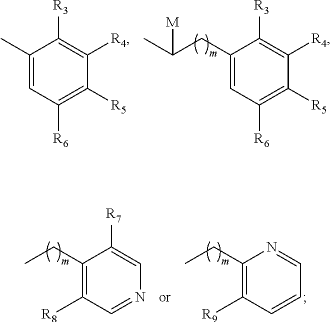

R₁, R₂, R₃, R₄, R₅, R₆, R₇, R₈, R₉ at each occurrence represent H, F, Cl, Br, —CN, —CH₃, —CF₃, —OCH₃, or —OCF₃; R₅ optionally represents phenyl, and M is H or —CH₃;

m represents a number of CH₂, and is 0 or 1;

n represents a number of CH₂, and is an integer from 1 to 10;

A is

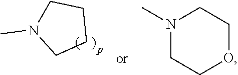

and p represents a number of CH₂, and is 1, 2, or 3; and

X is O or S.

The naphthylurea compound is one of the following compounds:

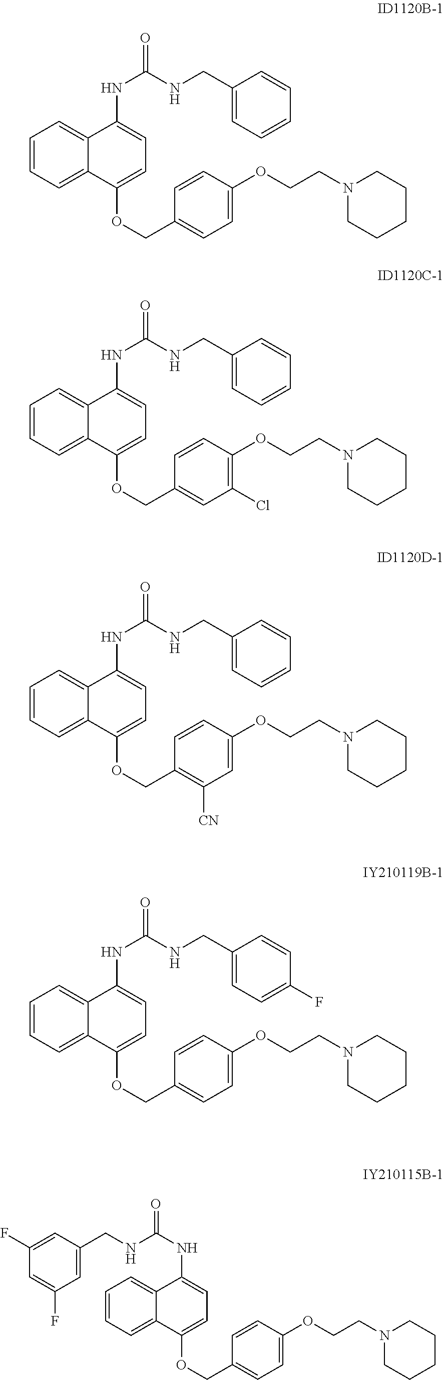 [SMILES: O=C(NCc1ccccc1)Nc1ccc(OCc2ccc(OCCN3CCCCC3)cc2)c2ccccc12 ; O=C(NCc1ccccc1)Nc1ccc(OCc2ccc(OCCN3CCCCC3)c(Cl)c2)c2ccccc12 ; N#Cc1cc(OCCN2CCCCC2)ccc1COc1ccc(NC(=O)NCc2ccccc2)c2ccccc12 ; O=C(NCc1ccc(F)cc1)Nc1ccc(OCc2ccc(OCCN3CCCCC3)cc2)c2ccccc12 ; O=C(NCc1cc(F)cc(F)c1)Nc1ccc(OCc2ccc(OCCN3CCCCC3)cc2)c2ccccc12]

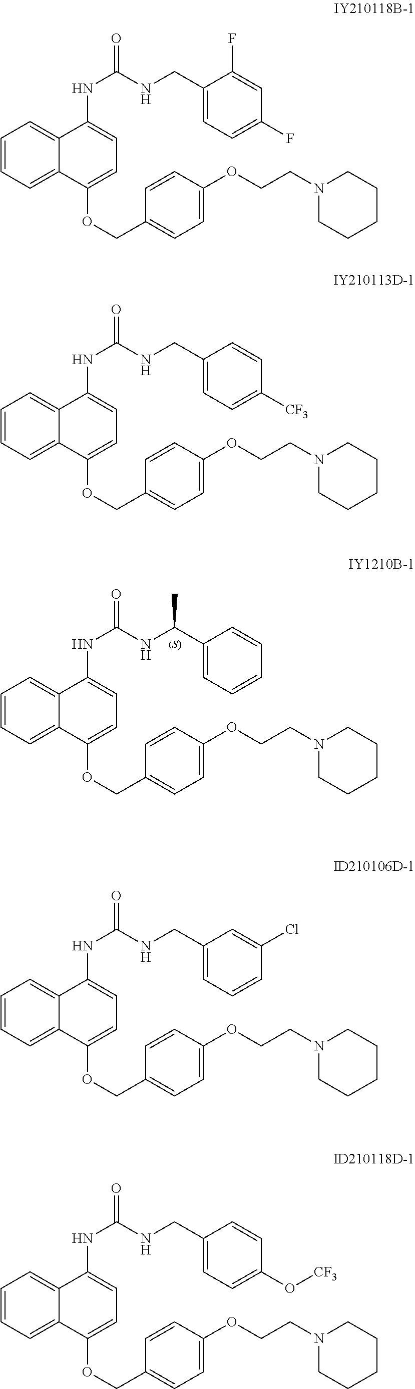

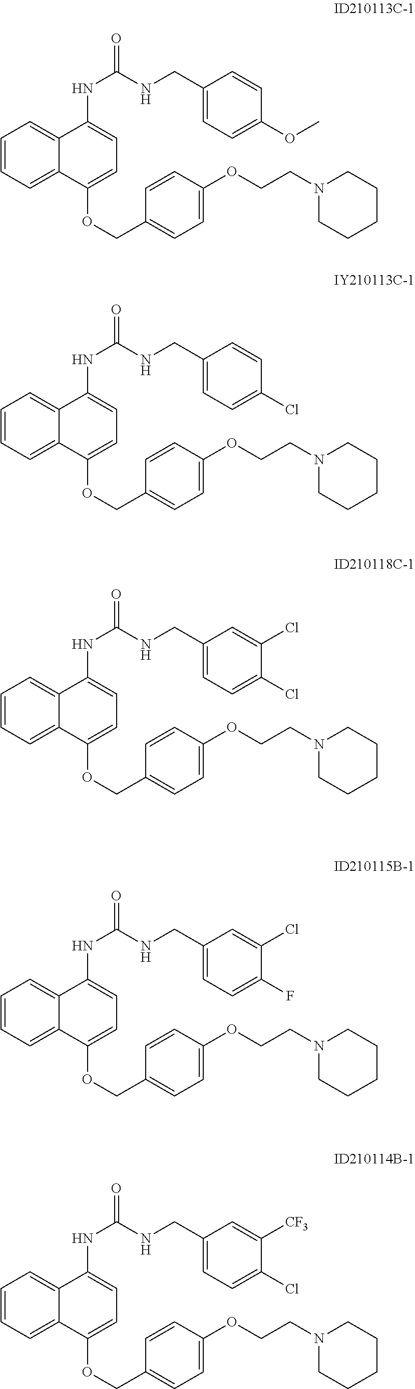 [SMILES: COc1ccc(CNC(=O)Nc2ccc(OCc3ccc(OCCN4CCCCC4)cc3)c3ccccc23)cc1 ; O=C(NCc1ccc(Cl)cc1)Nc1ccc(OCc2ccc(OCCN3CCCCC3)cc2)c2ccccc12 ; O=C(NCc1ccc(Cl)c(Cl)c1)Nc1ccc(OCc2ccc(OCCN3CCCCC3)cc2)c2ccccc12 ; O=C(NCc1ccc(F)c(Cl)c1)Nc1ccc(OCc2ccc(OCCN3CCCCC3)cc2)c2ccccc12 ; O=C(NCc1ccc(Cl)c(C(F)(F)F)c1)Nc1ccc(OCc2ccc(OCCN3CCCCC3)cc2)c2ccccc12]

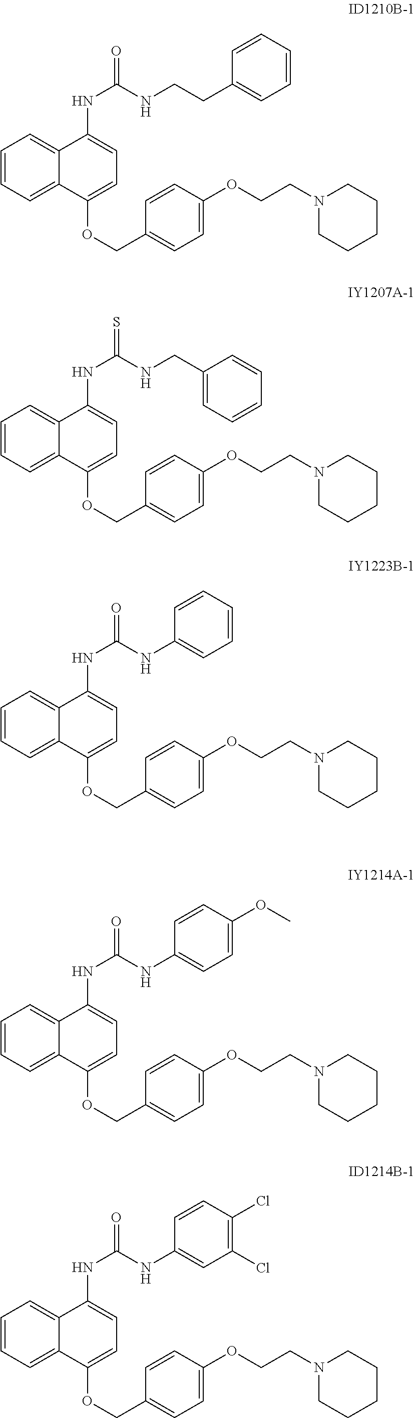 [SMILES: O=C(NCCc1ccccc1)Nc1ccc(OCc2ccc(OCCN3CCCCC3)cc2)c2ccccc12 ; S=C(NCc1ccccc1)Nc1ccc(OCc2ccc(OCCN3CCCCC3)cc2)c2ccccc12 ; O=C(Nc1ccccc1)Nc1ccc(OCc2ccc(OCCN3CCCCC3)cc2)c2ccccc12 ; COc1ccc(NC(=O)Nc2ccc(OCc3ccc(OCCN4CCCCC4)cc3)c3ccccc23)cc1 ; O=C(Nc1ccc(Cl)c(Cl)c1)Nc1ccc(OCc2ccc(OCCN3CCCCC3)cc2)c2ccccc12]

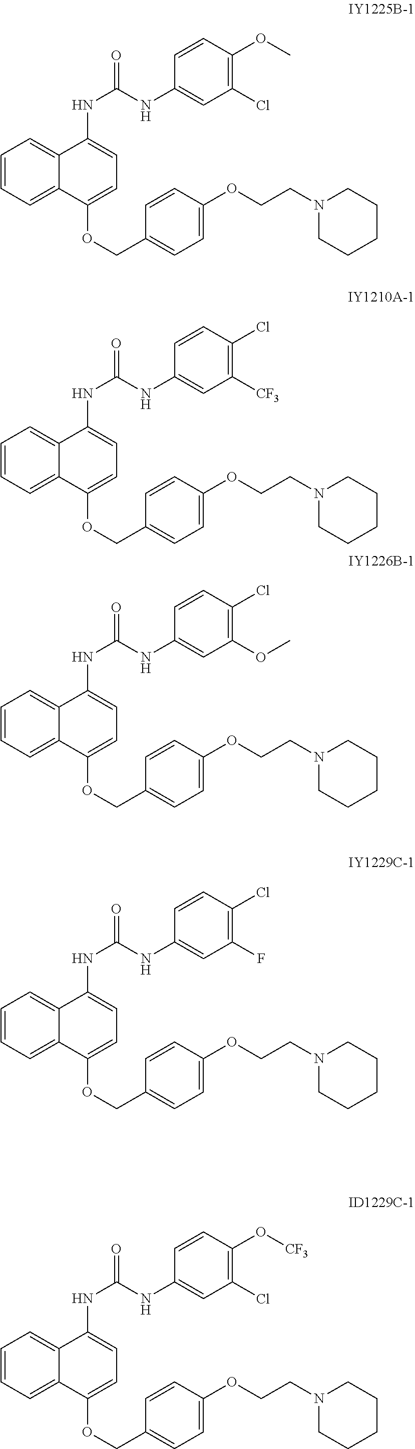 [SMILES: COc1ccc(NC(=O)Nc2ccc(OCc3ccc(OCCN4CCCCC4)cc3)c3ccccc23)cc1Cl ; O=C(Nc1ccc(Cl)c(C(F)(F)F)c1)Nc1ccc(OCc2ccc(OCCN3CCCCC3)cc2)c2ccccc12 ; COc1cc(NC(=O)Nc2ccc(OCc3ccc(OCCN4CCCCC4)cc3)c3ccccc23)ccc1Cl ; O=C(Nc1ccc(Cl)c(F)c1)Nc1ccc(OCc2ccc(OCCN3CCCCC3)cc2)c2ccccc12 ; O=C(Nc1ccc(OC(F)(F)F)c(Cl)c1)Nc1ccc(OCc2ccc(OCCN3CCCCC3)cc2)c2ccccc12]

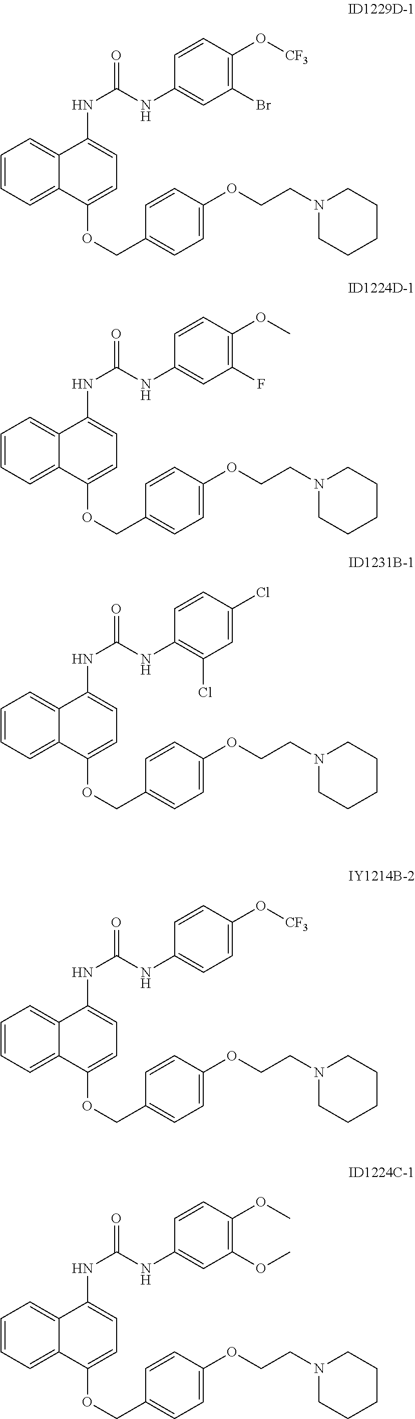 [SMILES: O=C(Nc1ccc(OC(F)(F)F)c(Br)c1)Nc1ccc(OCc2ccc(OCCN3CCCCC3)cc2)c2ccccc12 ; COc1ccc(NC(=O)Nc2ccc(OCc3ccc(OCCN4CCCCC4)cc3)c3ccccc23)cc1F ; O=C(Nc1ccc(Cl)cc1Cl)Nc1ccc(OCc2ccc(OCCN3CCCCC3)cc2)c2ccccc12 ; O=C(Nc1ccc(OC(F)(F)F)cc1)Nc1ccc(OCc2ccc(OCCN3CCCCC3)cc2)c2ccccc12 ; COc1ccc(NC(=O)Nc2ccc(OCc3ccc(OCCN4CCCCC4)cc3)c3ccccc23)cc1OC]

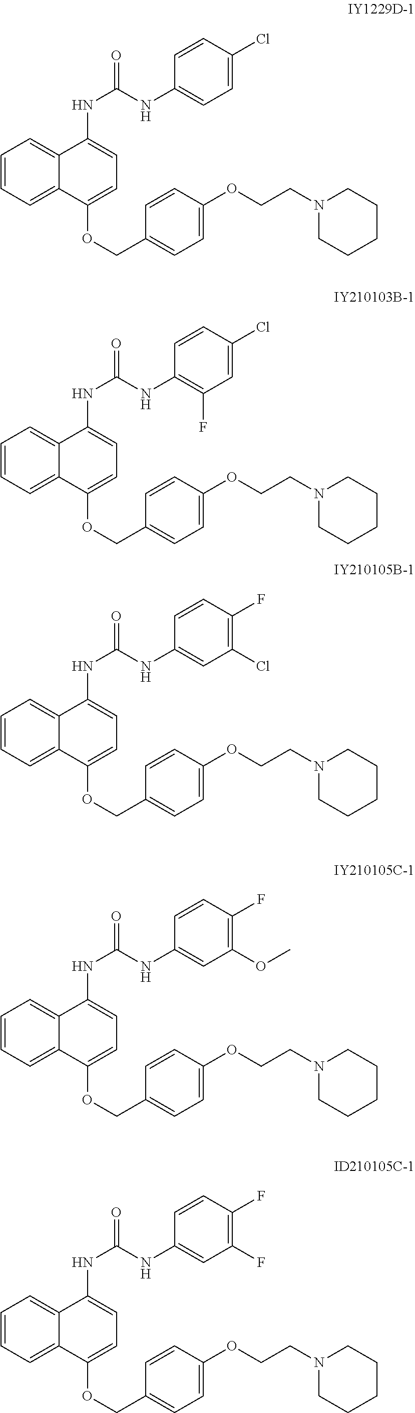 [SMILES: O=C(Nc1ccc(Cl)cc1)Nc1ccc(OCc2ccc(OCCN3CCCCC3)cc2)c2ccccc12 ; O=C(Nc1ccc(Cl)cc1F)Nc1ccc(OCc2ccc(OCCN3CCCCC3)cc2)c2ccccc12 ; O=C(Nc1ccc(F)c(Cl)c1)Nc1ccc(OCc2ccc(OCCN3CCCCC3)cc2)c2ccccc12 ; COc1cc(NC(=O)Nc2ccc(OCc3ccc(OCCN4CCCCC4)cc3)c3ccccc23)ccc1F ; O=C(Nc1ccc(F)c(F)c1)Nc1ccc(OCc2ccc(OCCN3CCCCC3)cc2)c2ccccc12]

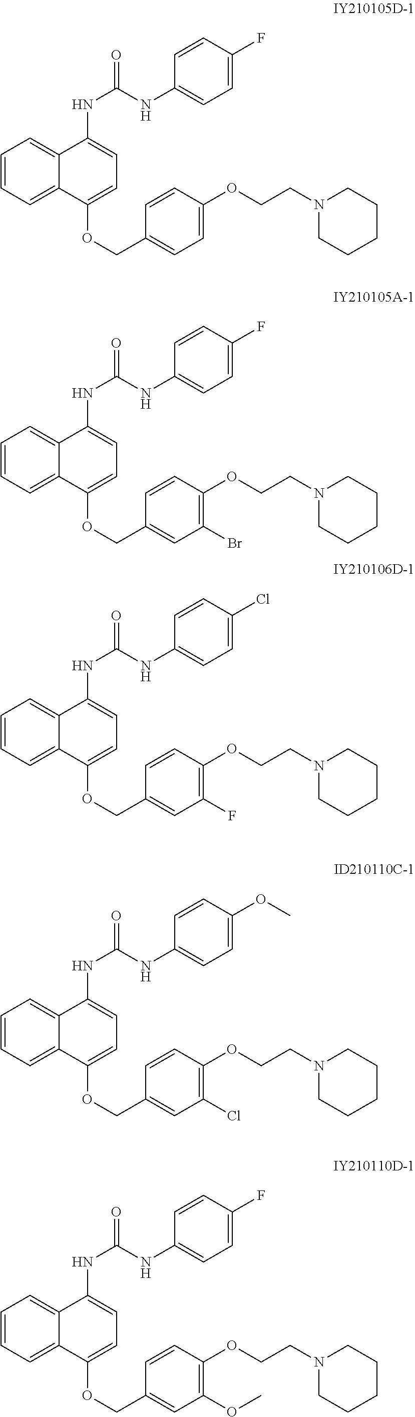 [SMILES: O=C(Nc1ccc(F)cc1)Nc1ccc(OCc2ccc(OCCN3CCCCC3)cc2)c2ccccc12 ; O=C(Nc1ccc(F)cc1)Nc1ccc(OCc2ccc(OCCN3CCCCC3)c(Br)c2)c2ccccc12 ; O=C(Nc1ccc(Cl)cc1)Nc1ccc(OCc2ccc(OCCN3CCCCC3)c(F)c2)c2ccccc12 ; COc1ccc(NC(=O)Nc2ccc(OCc3ccc(OCCN4CCCCC4)c(Cl)c3)c3ccccc23)cc1 ; COc1cc(COc2ccc(NC(=O)Nc3ccc(F)cc3)c3ccccc23)ccc1OCCN1CCCCC1]

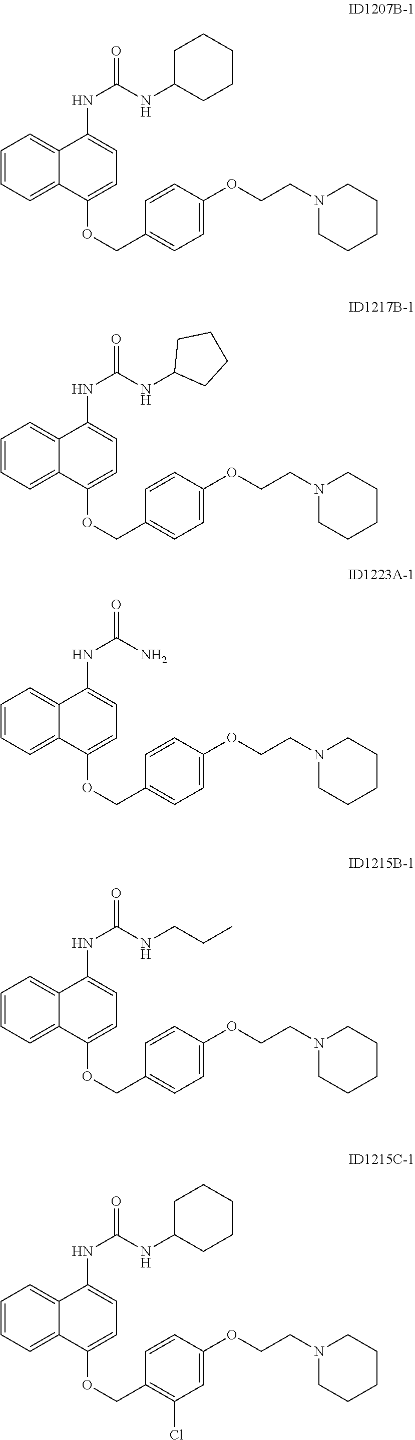 [SMILES: O=C(Nc1ccc(OCc2ccc(OCCN3CCCCC3)cc2)c2ccccc12)NC1CCCCC1 ; O=C(Nc1ccc(OCc2ccc(OCCN3CCCCC3)cc2)c2ccccc12)NC1CCCC1 ; NC(=O)Nc1ccc(OCc2ccc(OCCN3CCCCC3)cc2)c2ccccc12 ; CCCNC(=O)Nc1ccc(OCc2ccc(OCCN3CCCCC3)cc2)c2ccccc12 ; O=C(Nc1ccc(OCc2ccc(OCCN3CCCCC3)cc2Cl)c2ccccc12)NC1CCCCC1]

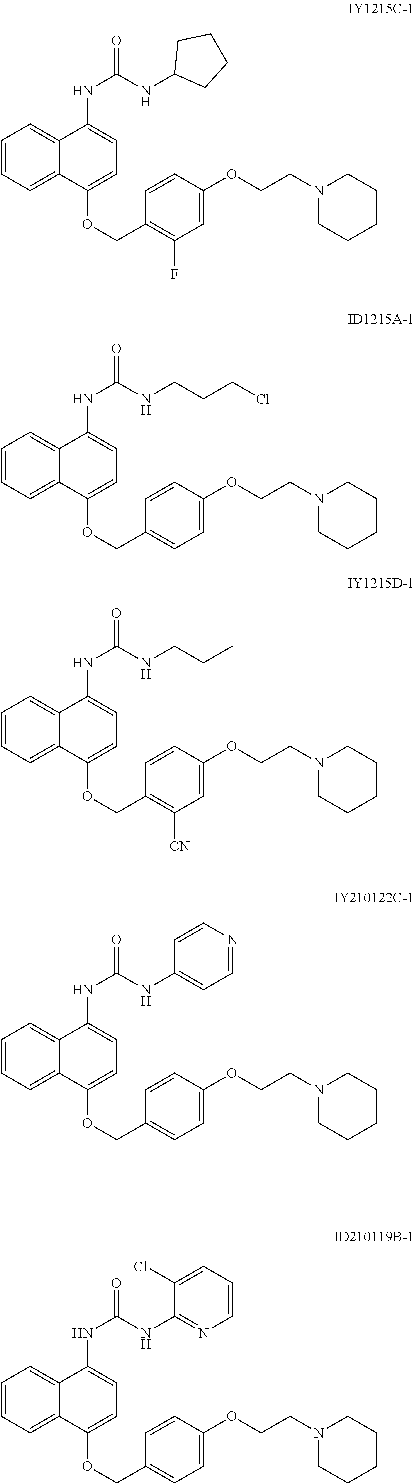 [SMILES: O=C(Nc1ccc(OCc2ccc(OCCN3CCCCC3)cc2F)c2ccccc12)NC1CCCC1 ; O=C(NCCCCl)Nc1ccc(OCc2ccc(OCCN3CCCCC3)cc2)c2ccccc12 ; CCCNC(=O)Nc1ccc(OCc2ccc(OCCN3CCCCC3)cc2C#N)c2ccccc12 ; O=C(Nc1ccncc1)Nc1ccc(OCc2ccc(OCCN3CCCCC3)cc2)c2ccccc12 ; O=C(Nc1ncccc1Cl)Nc1ccc(OCc2ccc(OCCN3CCCCC3)cc2)c2ccccc12]

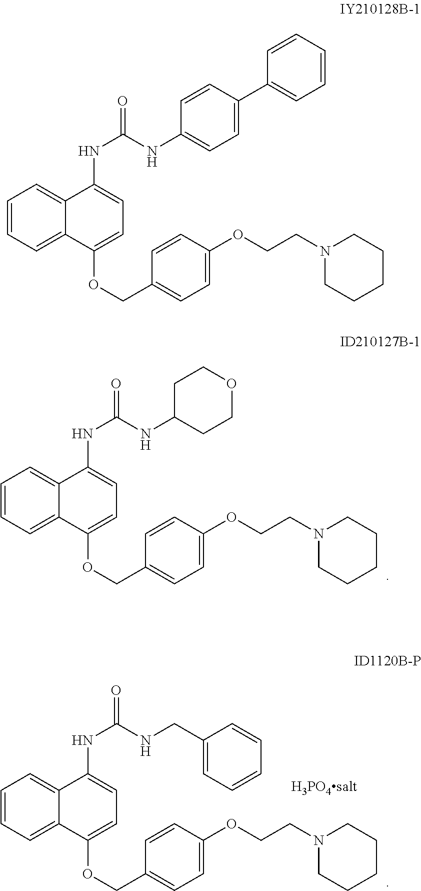 [SMILES: O=C(Nc1ccc(-c2ccccc2)cc1)Nc1ccc(OCc2ccc(OCCN3CCCCC3)cc2)c2ccccc12 ; O=C(Nc1ccc(OCc2ccc(OCCN3CCCCC3)cc2)c2ccccc12)NC1CCOCC1 ; O=C(NCc1ccccc1)Nc1ccc(OCc2ccc(OCCN3CCCCC3)cc2)c2ccccc12]

A biologically acceptable salt is formed by contacting the naphthylurea compound with at least an acid selected from the group consisting of acetic acid, dihydrofolic acid, benzoic acid, citric acid, sorbic acid, propionic acid, oxalic acid, fumaric acid, maleic acid, hydrochloric acid, malic acid, phosphoric acid, sulfite, sulfuric acid, vanillic acid, tartaric acid, ascorbic acid, boric acid, lactic acid, and ethylenediaminetetraacetic acid.

A method for preparing the naphthylurea compound comprises:

1) dissolving

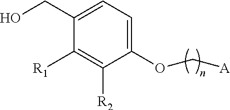

in tetrahydrofuran to yield a mixture, adding NaH in batches at −5-5° C. to the mixture, adding

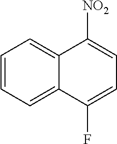 [SMILES: O=[N+]([O-])c1ccc(F)c2ccccc12]

to the mixture, and stirring at room temperature, to yield

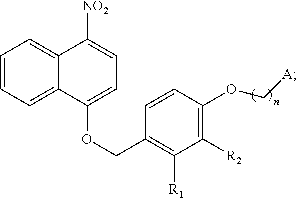

2) dissolving

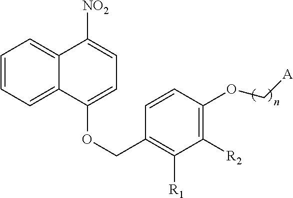

in a mixture solution of ethanol and saturated ammonium chloride aqueous solution, adding iron powders to the mixture solution at 40-50° C. and stirring at 50-60° C., to yield

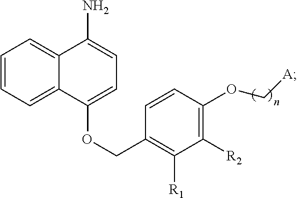

and

3) dissolving

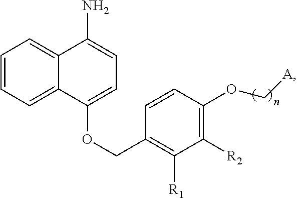

R-isocyanate or R-isothiocyanate, and N,N-diisopropylethylamine in 1,2-dichloroethane, stirring at 80-90° C., and extracting through column chromatography to yield

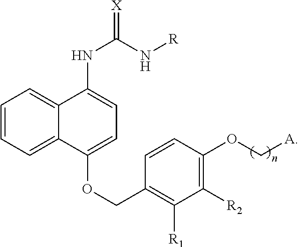

The compound

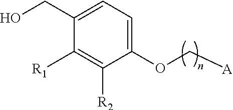

is prepared as follows:

(a) dissolving

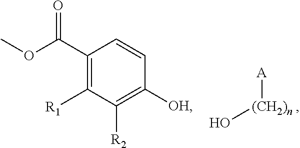

and triphenylphosphine in tetrahydrofuran, and adding diisopropyl azodicarboxylate to a resulting mixture at −5-5° C. under protective atmosphere, and stirring at room temperature, to yield

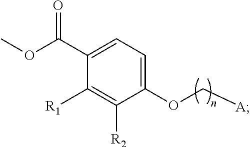

and

(b) dissolving

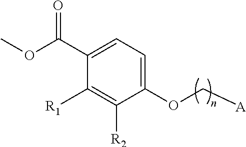

in tetrahydrofuran, adding lithium aluminum hydride in batches at −5-5° C., and stirring at room temperature, to yield

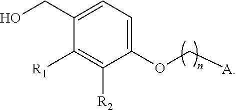

In 1), a molar ratio of

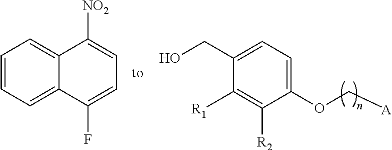

### to NaH is 1:1.2:2.

In 2), a molar ratio of

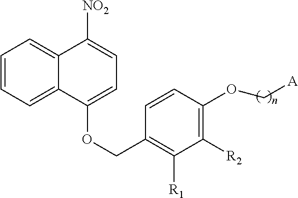

to the iron powders is 1:5, and a volume ratio of ethanol and the saturated ammonium chloride aqueous solution is 1:1.

In 3), a molar ratio of

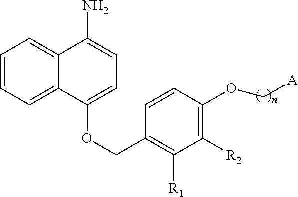

### to R-isocyanate or R-isothiocyanate, and to N,N-diisopropylethylamine is 1:1.2:2.0.

In a), a molar ratio of

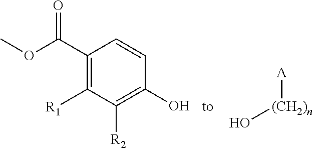

to triphenylphosphine to diisopropyl azodicarboxylate is 1:1.2:1.2:1.2; and in b), a molar ratio of

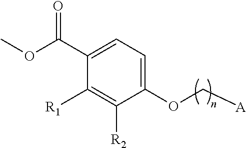

to lithium aluminum hydride is 1:1.

A method for treating a tumor comprises administering a patient in need thereof a naphthylurea compound of claim 1 or a biologically acceptable salt thereof.

Preferably, the tumor is liver cancer, breast cancer, lung cancer, or leukemia.

The second objective of the disclosure is to provide a small molecule compound with anti-tumor activity.

The tumor is highly proliferative or has a high level of CyclinB1 expression; the tumor includes, but is not limited to, liver cancer, breast cancer, lung cancer, leukemia, colon cancer, and lung cancer with resistant to tyrosine kinase inhibitor (TKI) therapy.

In a class of the embodiment, the compound ID1120B-1 and its derivatives ID1214B-1, IY1214A-1 and IY1214B-2 are synthesized; MTT assay is used to measure the anticancer activity of the compounds ID1120B-1, ID1214B-1, IY1214A-1 and IY1214B-2; flow cytometry is used to analyze the cell cycle and apoptosis of the tumor cells treated with the compounds ID1120B-1, ID1214B-1, IY1214A-1 and IY1214B-2.

The results indicate that the compounds ID1120B-1, ID1214B-1, IY1214A-1 and IY1214B-2 inhibit proliferation and development of tumor cells within liver cancer, breast cancer, lung cancer and leukemia, causing the tumor cells to be arrested in the G2/M phase of the cell cycle and undergo apoptosis.

### DETAILED DESCRIPTION OF THE DRAWINGS

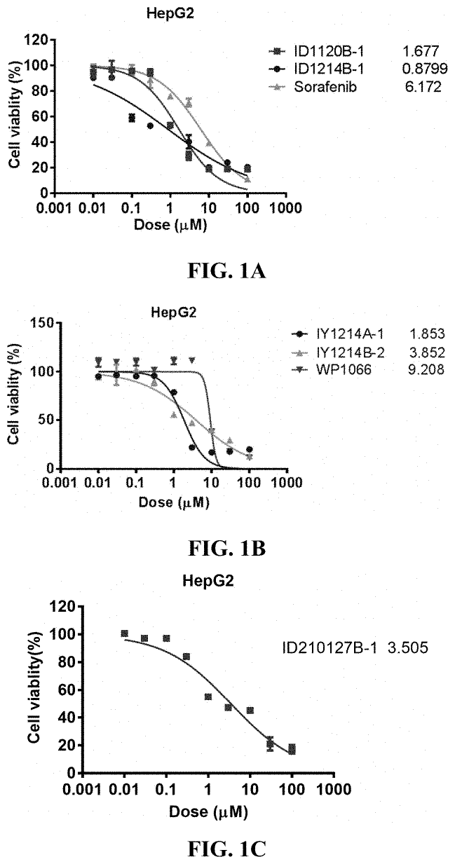

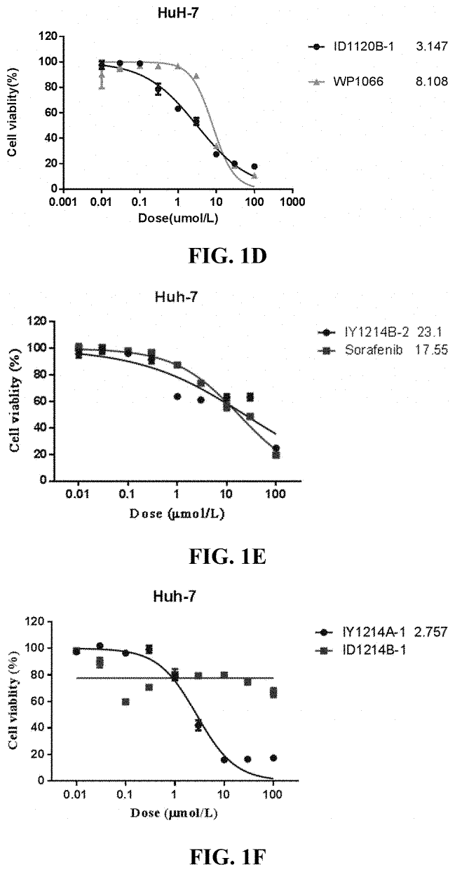

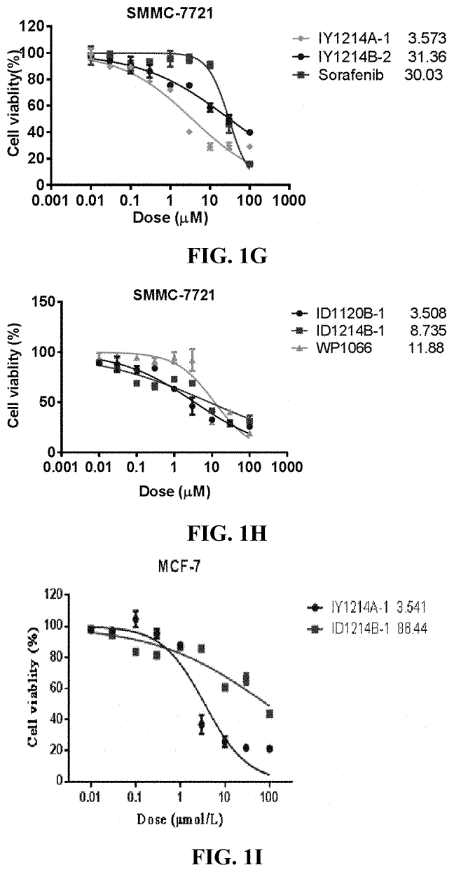

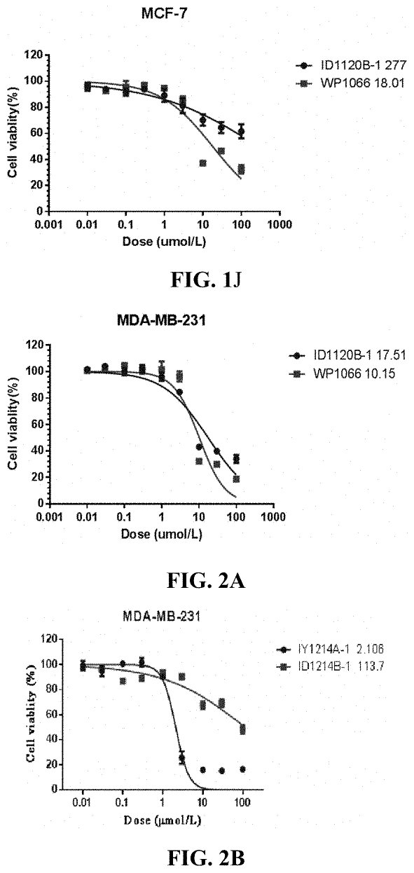

FIG. 1A shows the inhibitory effect of compounds ID1120B-1, ID1214B-1, and Sorafinib on the proliferation of HepG2 cells through MTT detection; the experimental results are expressed by IC50 (μM), and Sorafinib was used as a positive control drug;

FIG. 1B shows the inhibitory effect of compounds IY1214B-1, IY1214B-2, and WP1066 on the proliferation of hepatocellular carcinoma cell line HepG2 through MTT detection, and WP1066 is used as a control drug with the same target;

FIG. 1C shows the inhibitory effect of ID210127B-1 on the proliferation of HepG2 cells through MTT detection;

FIG. 1D shows the inhibitory effect of ID1120B-1 and WP1066 on the proliferation of liver cancer cell line HuH-7 through MTT detection, with WP1066 as a control drug with the same target;

FIG. 1E shows the inhibitory effect of IY1214B-2 and Sorafinib on the proliferation of liver cancer cell line HuH-7 through MTT detection, with Sorafinib as a positive control drug;

FIG. 1F shows the inhibitory effect of compounds IY1214A-1 and ID1214B-1 on the proliferation of hepatocellular carcinoma cell line HuH-7 through MTT detection;

FIG. 1G shows the inhibitory effect of compounds IY1214B-1, IY1214B-2, and Sorafinib on the proliferation of liver cancer cell line SMMC-7721 through MTT detection, with Sorafinib as a positive control drug;

FIG. 1H shows the inhibitory effect of compounds ID1120B-1, ID1214B-1, and WP1066 on the proliferation of liver cancer cell line SMMC-7721 through MTT detection, with WP1066 as a control drug with the same target;

FIG. 1I shows the inhibitory effect of compounds IY1214A-1 and ID1214B-1 on the proliferation of breast cancer cell MCF-7 through MTT detection;

FIG. 1J shows the inhibitory effect of compounds ID1120B-1 and WP1066 on the proliferation of breast cancer cell MCF-7 through MTT detection, with WP1066 as a control drug with the same target;

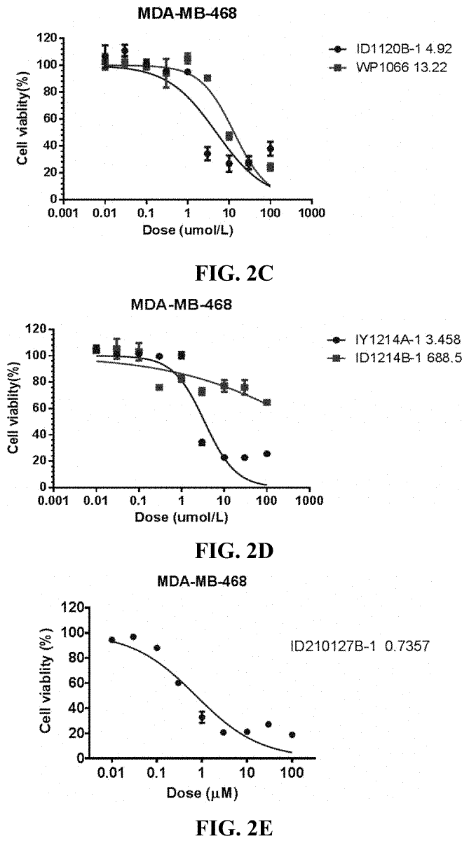

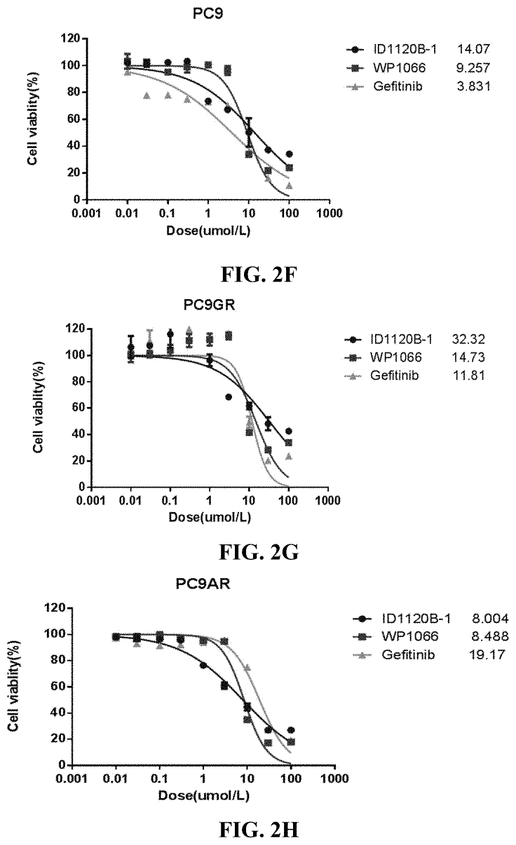

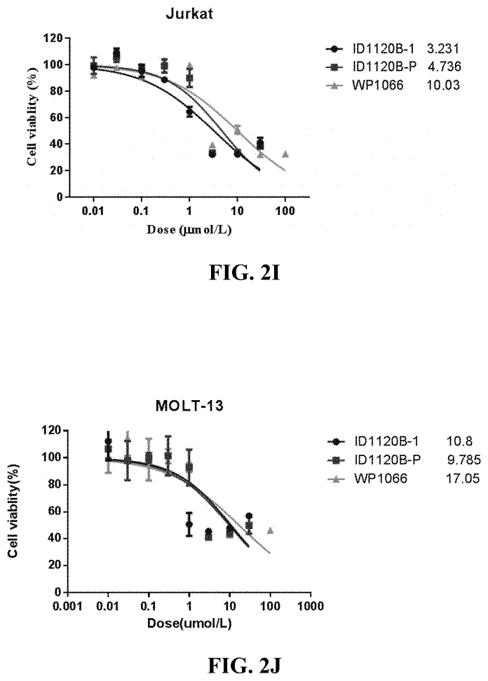

FIG. 2A shows the inhibitory effect of compounds ID1120B-1 and WP1066 on the proliferation of breast cancer cell MDA-MB-231 through MTT detection; WP1066 is used as the control drug of the same target;

FIG. 2B shows the inhibitory effect of IY1214A-1 and ID1214B-1 on the proliferation of breast cancer cell MDA-MB-231 through MTT detection;

FIG. 2C shows the inhibitory effect of compounds ID1120B-1 and WP1066 on the proliferation of breast cancer cell MDA-MB-468 through MTT detection; WP1066 is used as the control drug with the same target;

FIG. 2D shows the inhibitory effect of IY1214A-1 and ID1214B-1 on the proliferation of breast cancer cell line MDA-MB-468 through MTT detection;

FIG. 2E shows the inhibitory effect of ID210127B-1 on the proliferation of breast cancer cell MDA-MB-468 through MTT detection;

FIG. 2F shows the inhibitory effect of ID1120B-1, WP1066, and Gefitinib on the proliferation of lung cancer cell line PC9 through MTT detection; WP1066 is used as a control drug with the same target, and Gefitinib is used as a positive control drug for the same purpose;

FIG. 2G shows the inhibitory effect of ID1120B-1, WP1066, and Gefitinib on the proliferation of lung cancer drug resistant cell line PC9GR through MTT detection; WP1066 is used as a control drug with the same target, and Gefitinib is used as a positive control drug for the same purpose;

FIG. 2H shows the inhibitory effect of ID1120B-1, WP1066, and Gefitinib on the proliferation of lung cancer drug resistant cell line PC9AR through MTT detection; WP1066 is used as a control drug with the same target, and Gefitinib is used as a positive control drug for the same purpose;

FIG. 2I shows the inhibitory effect of ID1120B-1, ID1120B-P, and WP1066 on the proliferation of leukemia cell Jurkat through MTT detection, and WP1066 is used as a control drug with the same target;

FIG. 2J shows the inhibition effect of compounds ID1120B-1, ID1120B-P, and WP1066 on the proliferation of leukemia cell line MOLT-13 through MTT detection; WP1066 is used as a control drug with the same target;

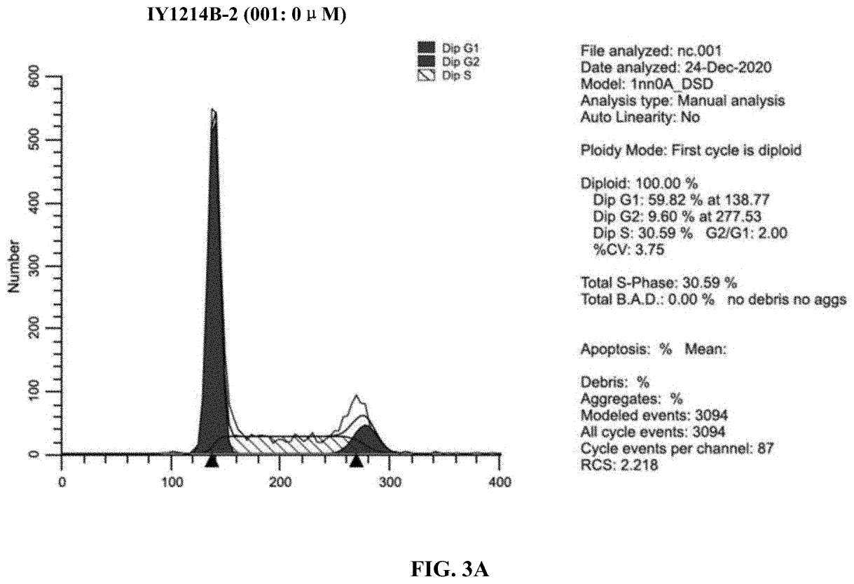

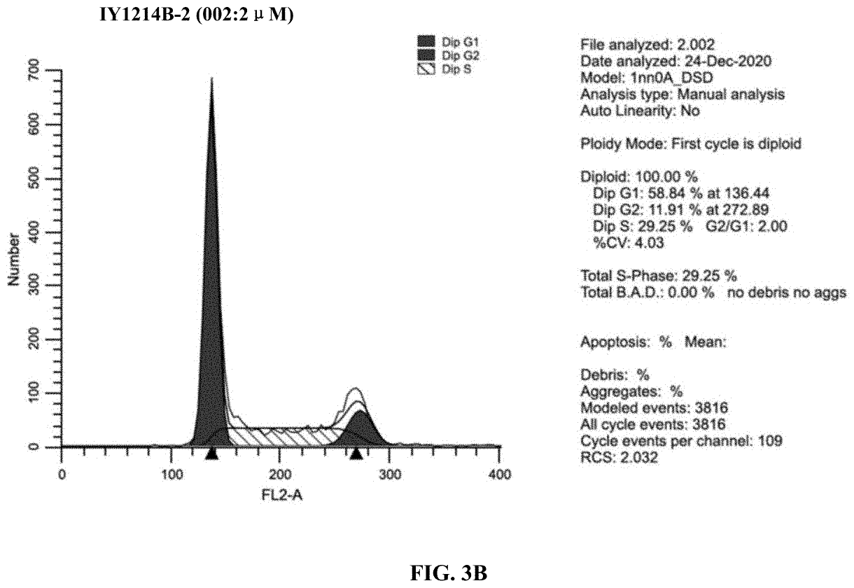

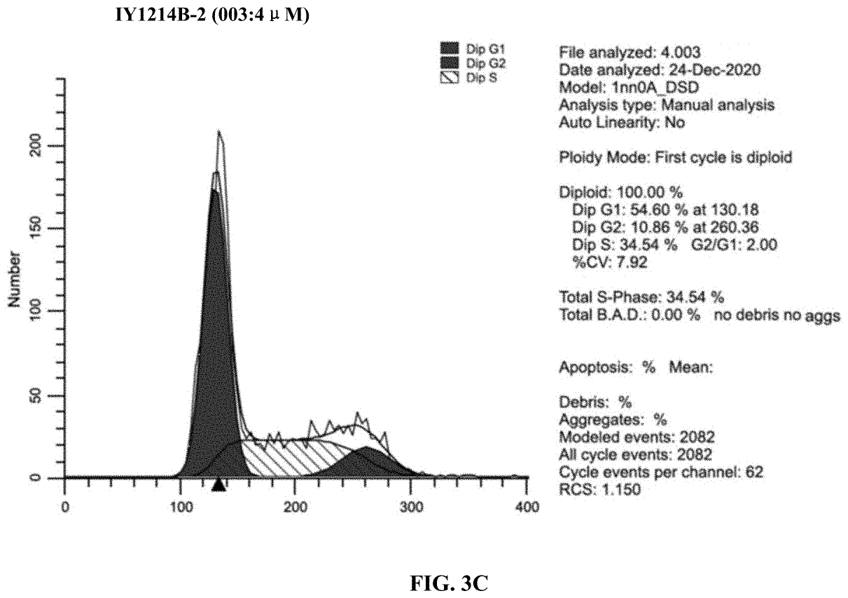

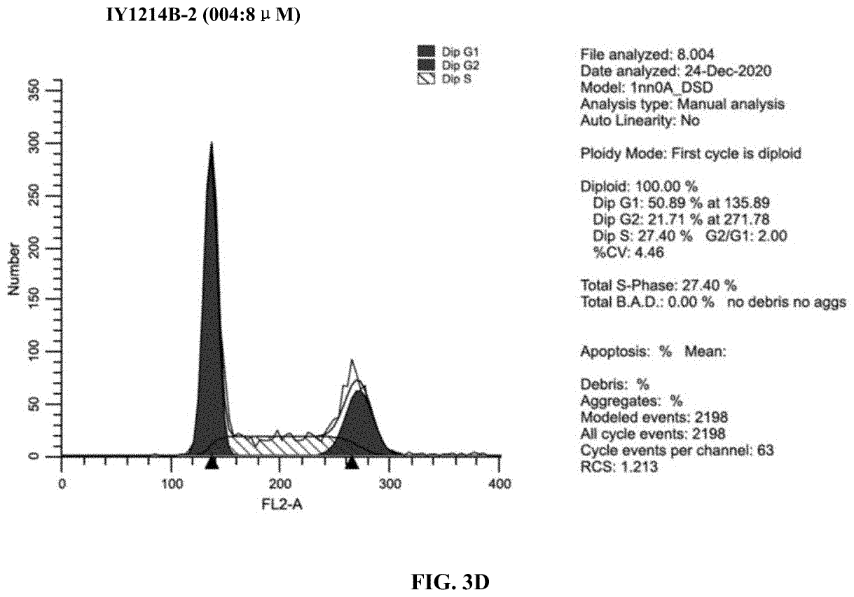

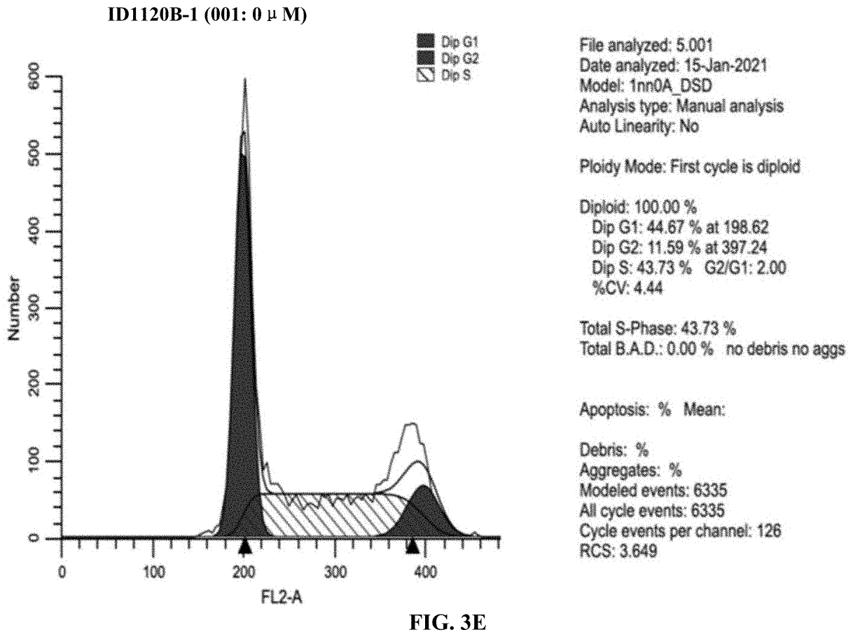

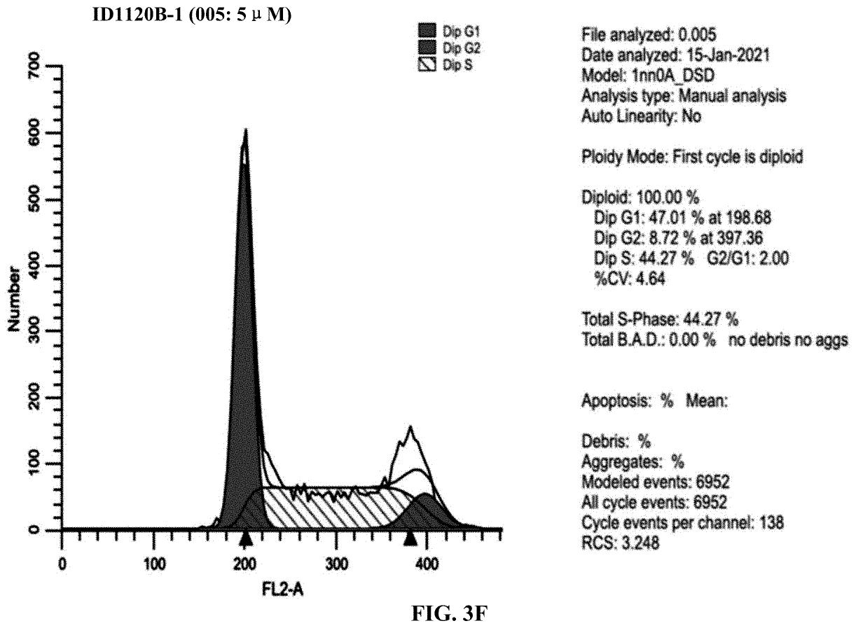

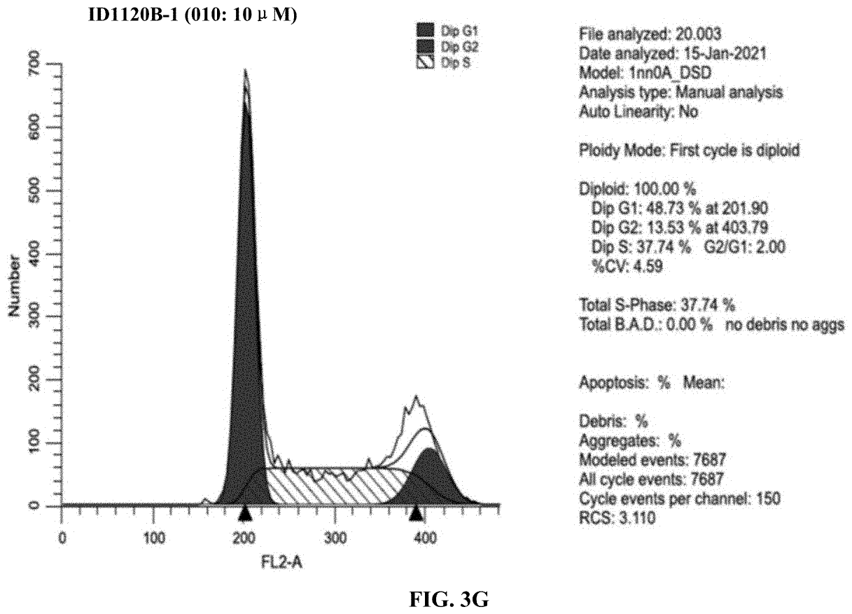

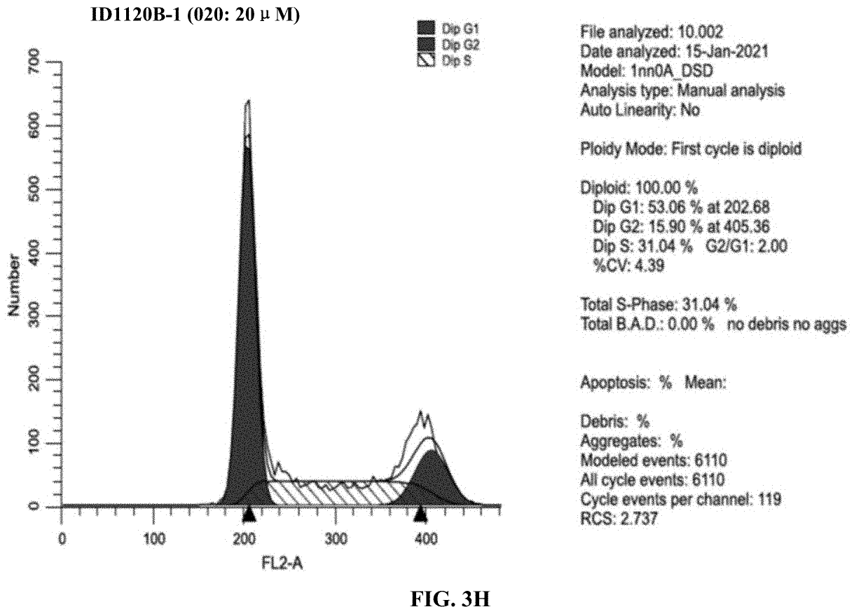

FIG. 3A shows the effect on the cell cycle of hepatocellular carcinoma cell line HepG2 after being treated by 0 μM compound IY1214B-2 for 48 hours;

FIG. 3B is the effect on the cell cycle of hepatocellular carcinoma cell line HepG2 after being treated by 2 μM compound IY1214B-2 for 48 hours;

FIG. 3C is the effect on the cell cycle of hepatocellular carcinoma cell line HepG2 after being treated by 4 μM compound IY1214B-2 for 48 hours;

FIG. 3D is the effect on the cell cycle of hepatocellular carcinoma cell line HepG2 after being treated by 8 μM compound IY1214B-2 for 48 hours;

FIG. 3E shows the effect on the cell cycle of hepatocellular carcinoma cell line HepG2 after being treated by 0 μM compound ID1120B-1 for 48 hours;

FIG. 3F is the effect on the cell cycle of hepatocellular carcinoma cell line HepG2 after being treated by 5 μM compound ID1120B-1 for 48 hours;

FIG. 3G is the effect on the cell cycle of hepatocellular carcinoma cell line HepG2 after being treated by 10 μM compound ID1120B-1 for 48 hours;

FIG. 3H is the effect on the cell cycle of hepatocellular carcinoma cell line HepG2 after being treated by 20 μM compound ID1120B-1 for 48 hours;

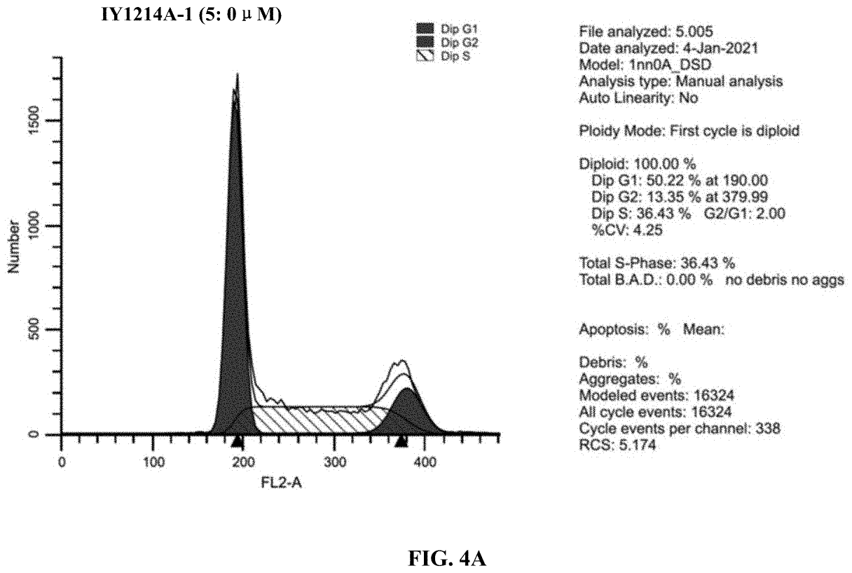

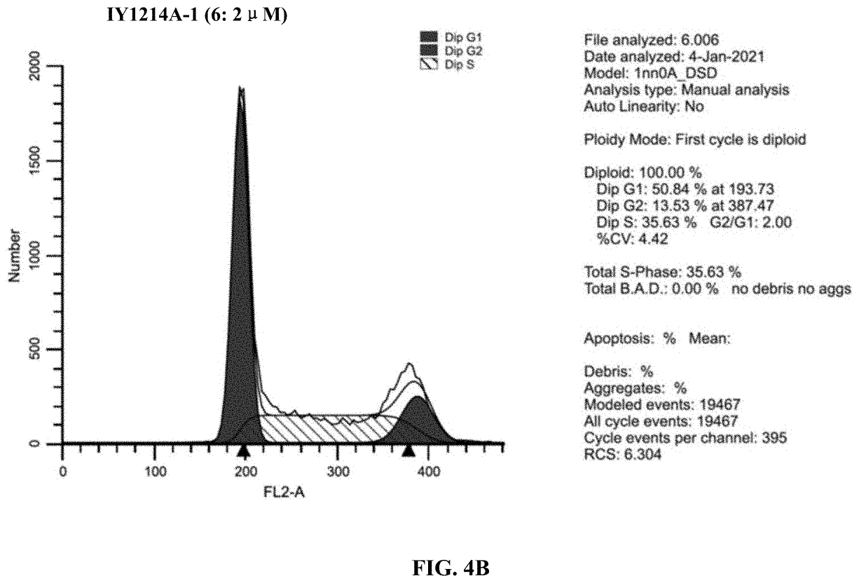

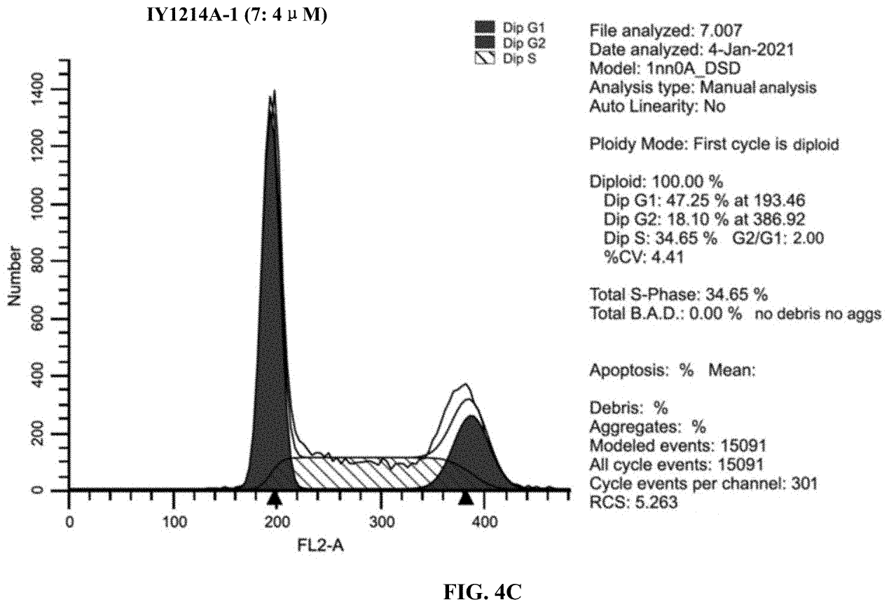

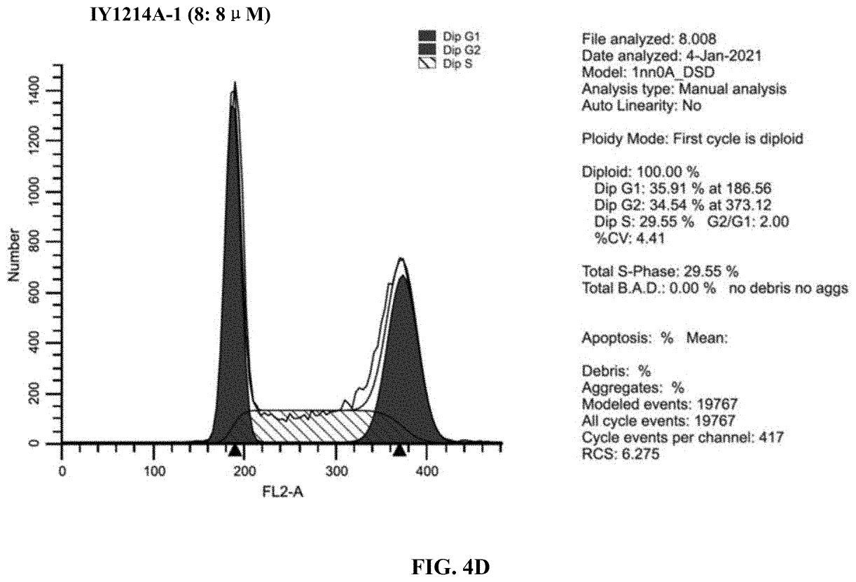

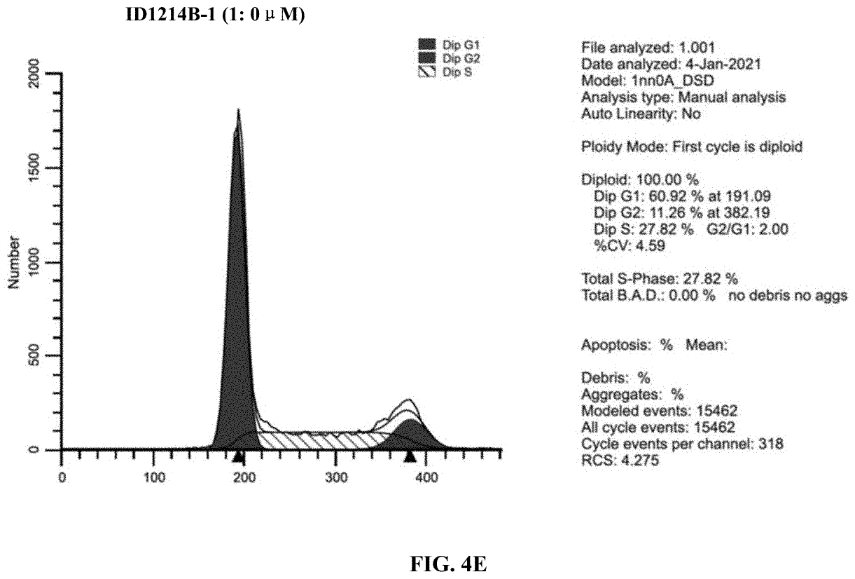

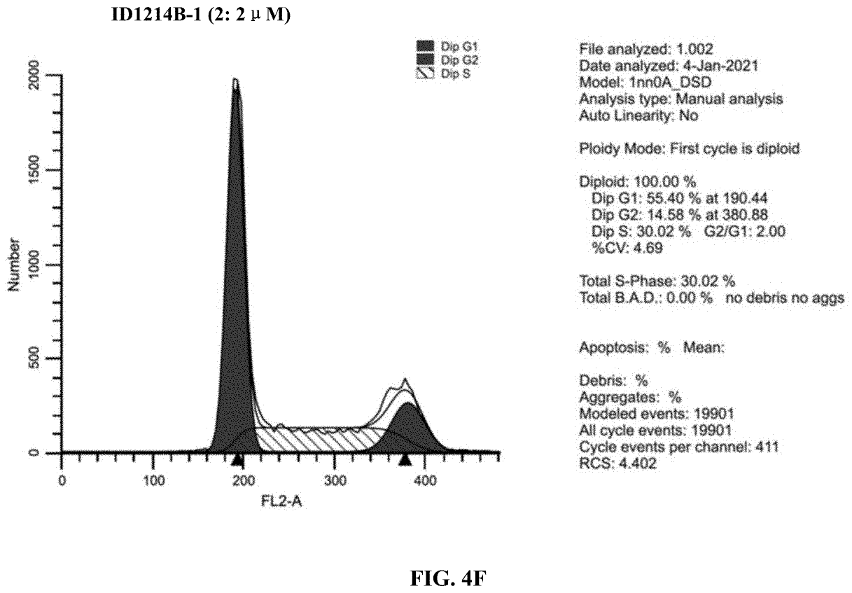

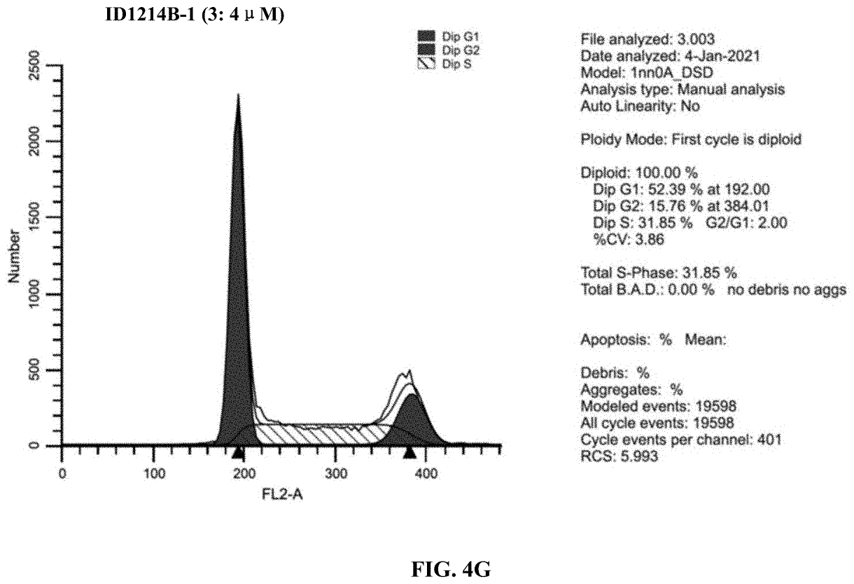

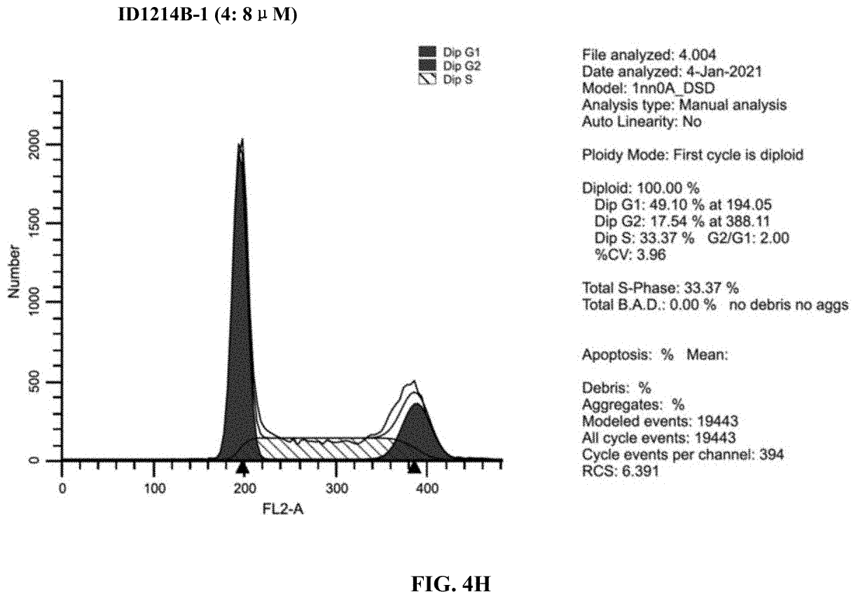

FIG. 4A shows the effect on the cell cycle of hepatocellular carcinoma cell line HepG2 after being treated by 0 μM compound IY1214A-1 for 48 hours;

FIG. 4B is the effect on the cell cycle of hepatocellular carcinoma cell line HepG2 after being treated by 2 μM compound IY1214A-1 for 48 hours;

FIG. 4C is the effect on the cell cycle of hepatocellular carcinoma cell line HepG2 after being treated by 4 μM compound IY1214A-1 for 48 hours;

FIG. 4D is the effect on the cell cycle of hepatocellular carcinoma cell line HepG2 after being treated by 8 μM compound IY1214A-1 for 48 hours;

FIG. 4E shows the effect on the cell cycle of hepatocellular carcinoma cell line HepG2 after being treated by 0 μM compound ID1214B-1 for 48 hours;

FIG. 4F is the effect on the cell cycle of hepatocellular carcinoma cell line HepG2 after being treated by 2 μM compound ID1214B-1 for 48 hours;

FIG. 4G is the effect on the cell cycle of hepatocellular carcinoma cell line HepG2 after being treated by 4 μM compound ID1214B-1 for 48 hours;

FIG. 4H is the effect on the cell cycle of hepatocellular carcinoma cell line HepG2 after being treated by 8 μM compound ID1214B-1 for 48 hours;

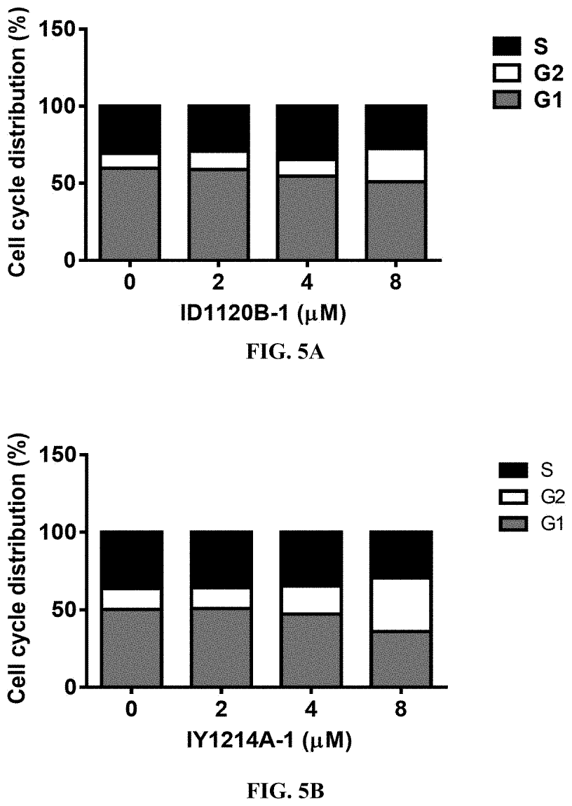

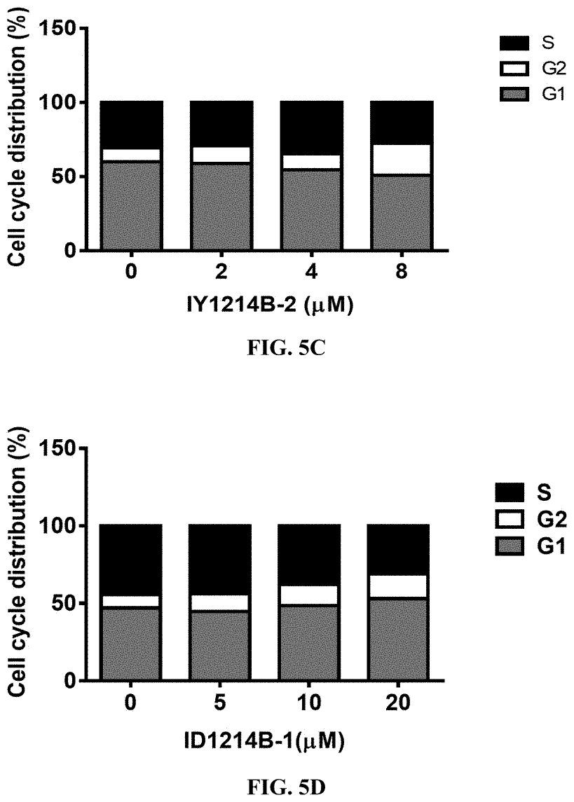

FIG. 5A is statistical analysis of the results of FIGS. 3E-3G;

FIG. 5B is statistical analysis of the results of FIGS. 4A-4D;

FIG. 5C is statistical analysis of the results of FIGS. 3A-3D;

FIG. 5D is statistical analysis of the results of FIGS. 4E-4H;

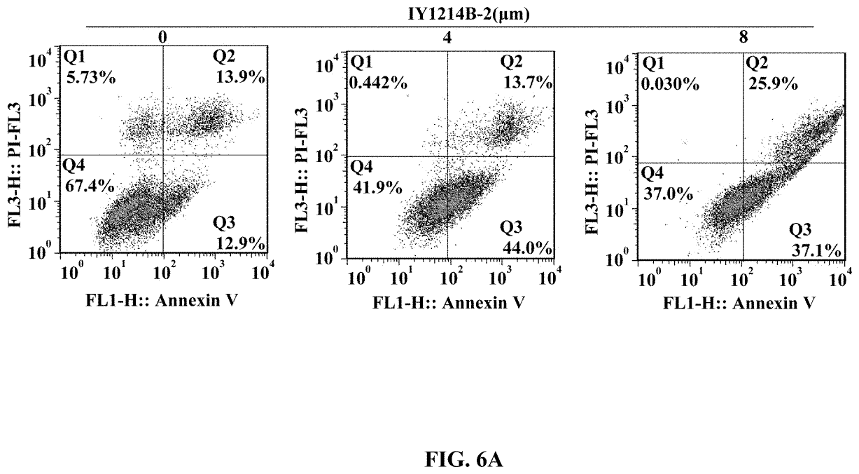

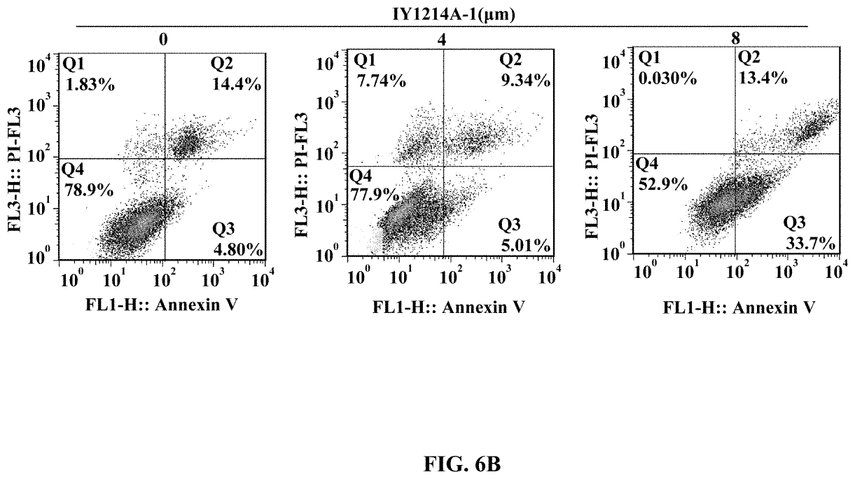

FIG. 6A shows the effect on apoptosis of HepG2 cells after being treated by 0, 4, and 8 μM compound IY1214B-2 for 48 hours through flow cytometry detection;

FIG. 6B shows the effect on apoptosis of HepG2 cells after being treated by 0, 4, and 8 μM compound IY1214A-1 for 48 hours through flow cytometry detection; and

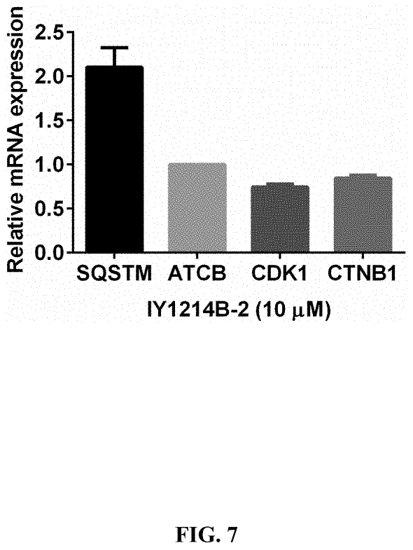

FIG. 7 shows qPCR results of regulation of mRNA expression levels of cell cycle regulatory molecules and autophagy-related genes by a compound IY1214B-2.

### DETAILED DESCRIPTION

To further illustrate the disclosure, embodiments detailing a naphthylurea compound are described below. It should be noted that the following embodiments are intended to describe and not to limit the disclosure.

In a method for synthesizing the naphthylurea compound having the formula I, all raw materials are commercially available or prepared by those skilled in the prior arts. In the disclosure, the intermediates, raw materials, reagents, and reaction conditions are changed by the person skilled in the art.

In the disclosure, (i) the temperature is seen in units of degree Celsius or ° C.; and the synthesis method is performed at room temperature ranging from 20° C. to 30° C.; (ii) a common method is used to dry the organic solvent; a rotary evaporator is used to remove solvent from a sample through evaporation under reduced pressure; the maximum temperature for a bath is 50° C.; a developing solvent and an eluting solvent are added in a volume ratio; (iii) thin layer chromatography (TLC) is used to monitor the progress of chemical reaction; (iv) a final product is obtained and produces enough signals in a 1H NMR spectrum.

### Example 1 Compound Synthesis

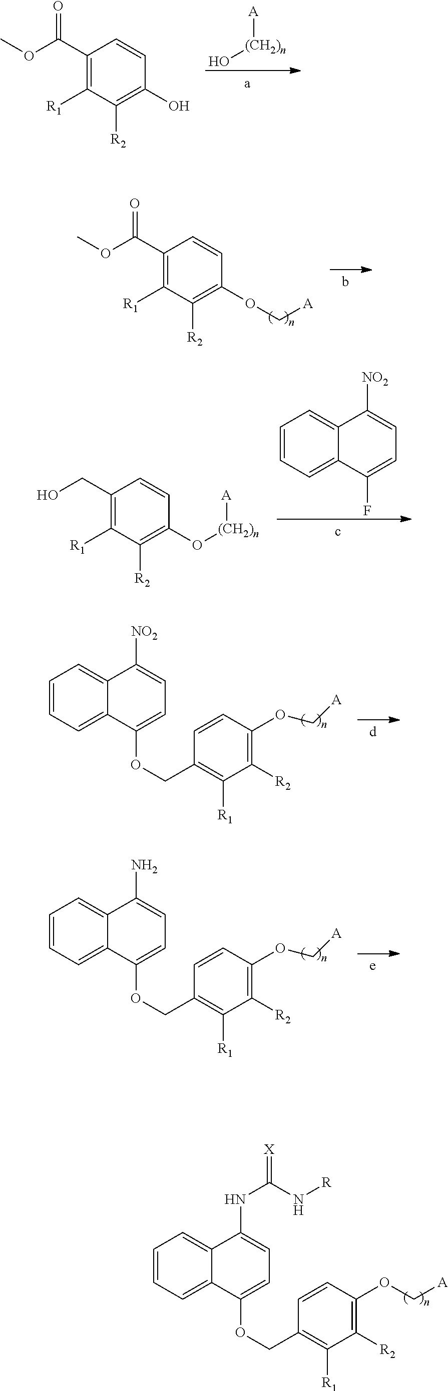

ID1120B-1: R═

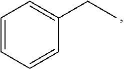

R₁═H, R₂═H, n=2, A=

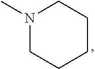

### X═O;

ID1120C-1: R═

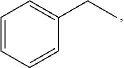

R₁═H, R₂═Cl, n=2, A=

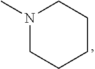

### X═O;

ID1120D-1: R═

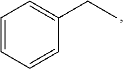

R₁═CN, R₂═H, n=2, A=

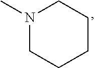

### X═O;

IY210119B-1: R═

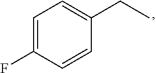

R₁═H, R₂═H, n=2, A=

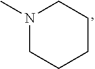

### X═O;

IY210115B-1: R═

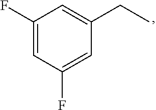

R₁═H, R₂═H, n=2, A=

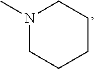

### X═O;

IY210118B-1: R═

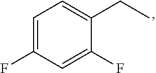

R₁═H, R₂═H, n=2, A=

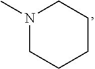

### X═O;

IY210113D-1: R═

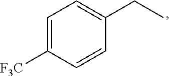

R₁═H, R₂═H, n=2, A=

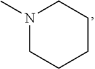

### X═O;

IY1210B-1: R═

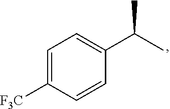

R₁═H, R₂═H, n=2, A=

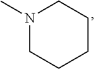

### X═O;

ID210106D-1: R═

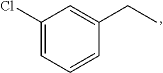

R₁═H, R₂═H, n=2, A=

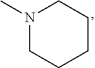

### X═O;

ID210118D-1: R═

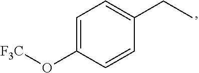

R₁═H, R₂═H, n=2, A=

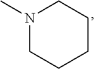

### X═O;

ID210113C-1: R═

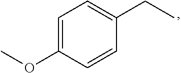

R₁═H, R₂═H, n=2, A=

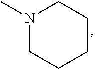

### X═O;

IY210113C-1: R═

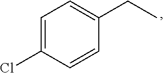

R₁═H, R₂═H, n=2, A=

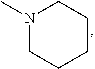

### X═O;

ID210118C-1: R═

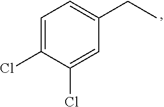

R₁═H, R₂═H, n=2, A=

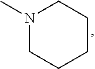

### X═O;

ID210115B-1: R═

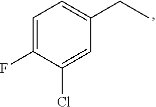

R₁═H, R₂═H, n=2, A=

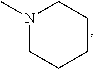

### X═O;

ID210114B-1: R═

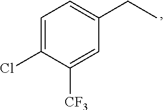

R₁═H, R₂═H, n=2, A=

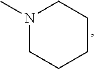

### X═O;

ID1210B-1: R═

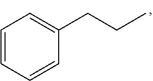

R₁═H, R₂═H, n=2, A=

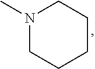

### X═O;

IY1207A-1: R═

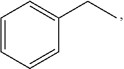

RI=H, R₂═H, n=2, A=

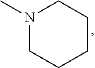

### X═S;

IY1223B-1: R═

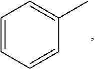

R₁═H, R₂═H, n=2, A=

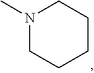

### X═O;

IY1214A-1: R═

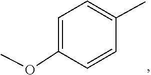

R₁═H, R₂═H, n=2, A=

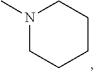

### X═O;

ID1214B-1: R═

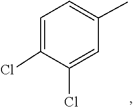

R₁═H, R₂═H, n=2, A=

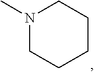

### X═O;

IY1225B-1: R═

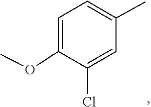

R₁═H, R₂═H, n=2, A=

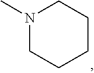

### X═O;

IY1210A-1: R═

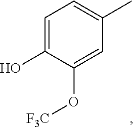

R₁═H, R₂═H, n=2, A=

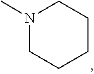

### X═O;

IY1226B-1: R═

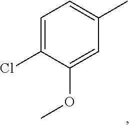

R₁═H, R₂═H, n=2, A=

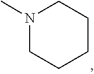

### X═O;

IY1229C-1: R═

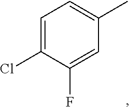

R₁═H, R₂═H, n=2, A=

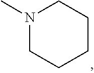

### X═O;

ID1229C-1: R═

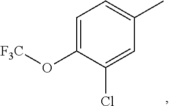

R₁═H, R₂═H, n=2, A=

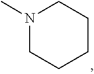

### X═O;

ID1229D-1: R═

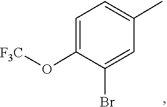

R₁═H, R₂═H, n=2, A=

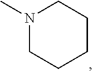

### X═O;

ID1224D-1: R═

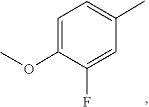

R₁═H, R₂═H, n=2, A=

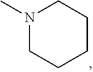

### X═O;

ID1231B-1: R═

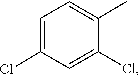

R₁═H, R₂═H, n=2, A=

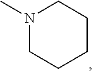

### X═O;

IY1214B-2: R═

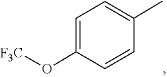

R₁═H, R₂═H, n=2, A=

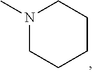

### X═O;

ID1224C-1: R═

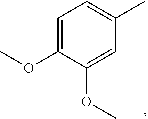

R₁═H, R₂═H, n=2, A=

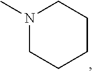

### X═O;

IY1229D-1: R═

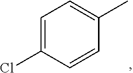

R₁═H, R₂═H, n=2, A=

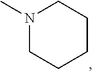

### X═O;

IY210103B-1: R═

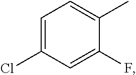

R₁═H, R₂═H, n=2, A=

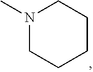

### X═O;

IY210105B-1: R═

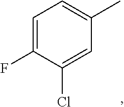

R₁═H, R₂═H, n=2, A=

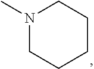

### X═O;

IY210105C-1: R═

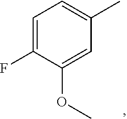

R₁═H, R₂═H, n=2, A=X═O;

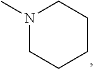

### X═O;

ID210105C-1: R═

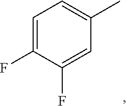

R₁═H, R₂═H, n=2, A=

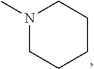

### X═O;

IY210105D-1: R═

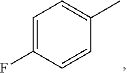

R₁═H, R₂═H, n=2, A=

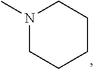

### X═O;

IY210105A-1: R═

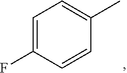

R₁═H, R₂═Br, n=2, A=

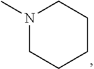

### X═O;

IY210106D-1: R═

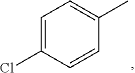

R₁═H, R₂═F, n=2, A=

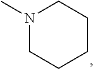

### X═O;

ID210110C-1: R═

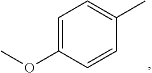

R₁═H, R₂═Cl, n=2, A=

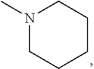

### X═O;

IY210110D-1: R═

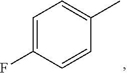

R₁═H, R₂═OMe, n=2, A=

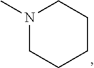

### X═O;

ID1207B-1: R═

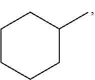

R₁═H, R₂═H, n=2, A=

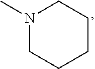

### X═O;

ID1217B-1: R═

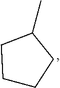

R₁═H, R₂═H, n=2, A=

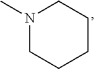

### X═O;

ID1223A-1: R═H, R₁═H, R₂═H, n=2, A=

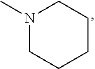

### X═O;

ID1215B-1: R═

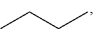

R₁═H, R₂═H, n=2, A=

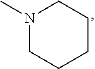

### X═O;

ID1215C-1: R═

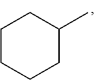

R₁═Cl, R₂═H, n=2, A=

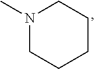

### X═O;

IY1215C-1: R═

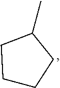

R₁═F, R₂═H, n=2, A=

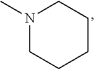

### X═O;

ID1215A-1: R═

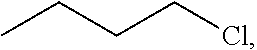

R₁═H, R₂═H, n=2, A=

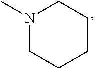

### X═O;

IY1215D-1: R═

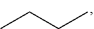

R₁═CN, R₂═H, n=2, A=

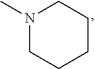

### X═O;

IY210122C-1: R═

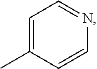

R₁═H, R₂═H, n=2, A=

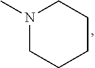

### X═O;

ID210119B-1: R═

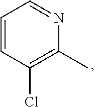

R₁═H, R₂═H, n=2, A=

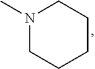

### X═O;

IY210128B-1: R═

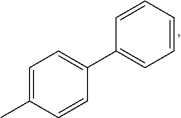

R₁═H, R₂═H, n=2, A=

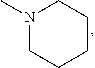

### X═O;

ID210127B-1: R═

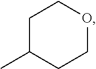

R₁═H, R₂═H, n=2, A=

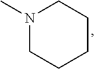

### X═O;

For example, the naphthylurea compound ID 1120B-1 and a phosphate ID 1120B-P thereof respectively having the following two formulas:

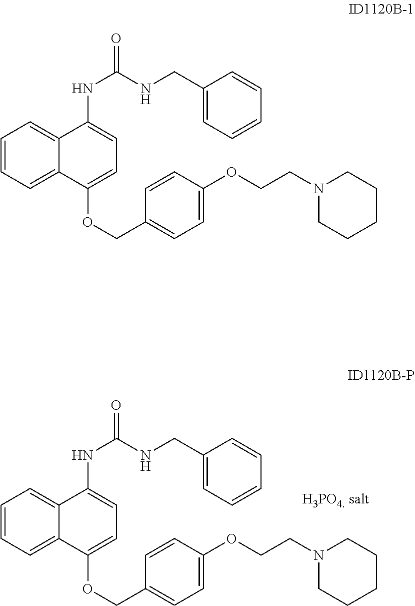 [SMILES: O=C(NCc1ccccc1)Nc1ccc(OCc2ccc(OCCN3CCCCC3)cc2)c2ccccc12 ; O=C(NCc1ccccc1)Nc1ccc(OCc2ccc(OCCN3CCCCC3)cc2)c2ccccc12]

The naphthylurea compound ID1120B-1 is named 1-benzyl-3-(4-((4-(2-(piperidin-1-yl)ethoxy)benzyl)oxy)naphthalen-1-yl)urea.

The naphthylurea compound ID1120B-1 is synthesized by the following route:

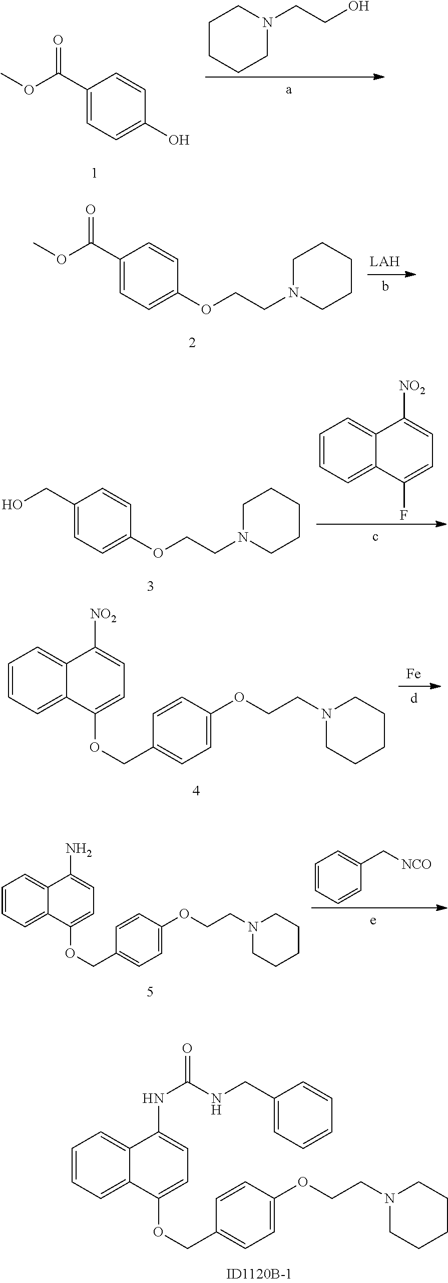

### Step 1. Preparation of methyl 4-(2-(piperidin-1-yl)ethoxy)benzoate (Compound 2)

1.0 g of methyl 4-hydroxybenzoate (Compound 1, 6.57 mmol, 1.0 eq), 1.02 g of N-hydroxyethylpiperidine (7.89 mmol, 1.2 eq) and 2.07 g of triphenylphosphine (7.89 mmol, 1.2 eq) was dissolved in 30 mL of anhydrous tetrahydrofuran (THF) to yield a mixture; the mixture was cooled to 0° C.; 1.59 g of diisopropyl azodicarboxylate (7.89 mmol, 1.2 eq) was added dropwise to the cooled mixture under nitrogen and allowed to react at room temperature for 16 h; when a thin layer chromatography (TLC) plate showed that no more starting materials are left in the reaction time, the resulting mixture was concentrated under reduced pressure to remove THF, and a solid is formed; the solid was dissolved in ethyl acetate to form a solution; the pH of the solution was adjusted to 1 with 1N hydrochloric acid; the solution was extracted three times with ethyl acetate; the pH of the aqueous phase was adjusted to 8 with sodium bicarbonate; the aqueous phase was extracted three times with ethyl acetate; the organic phase was dried and spin-dried to yield 1.5 g of a white solid; the white solid is methyl 4-(2-(piperidin-1-yl)ethoxy)benzoate (Compound 2) in 86.7% yield).

1H NMR(CDCl3, 300 MHz) δ: 8.0 (d, J=9.0 Hz, 2H), 6.93 (d, J=9.0 Hz, 2H), 4.17 (t, J=6.0 Hz, 2H), 3.90 (s, 3H), 2.82 (t, J=6.0 Hz, 2H), 2.58-2.55 (m, 4H), 1.66-1.61 (m, 4H), 1.50 (t, J=3.0 Hz, 2H)

### Step 2. Preparation of (4-(2-(piperidin-1-yl)ethoxy)phenyl)methanol (Compound 3)

1.00 g of methyl 4-(2-(piperidin-1-yl)ethoxy)benzoate (Compound 2, 3.80 mmol, 1.0 eq) was dissolved in 40 mL of anhydrous THF to yield a solution; the solution was cooled to 0° C.; 144 mg of lithium aluminum hydride (3.80 mmol, 1.0 eq) was added in batches to the cooled solution to form a mixture; the mixture temperature was naturally raised to room temperature and the mixture was allowed to react at room temperature for 0.5 h; the TLC plate showed that no more starting materials were left in the reaction mixture and new spots were visualized; the reaction mixture was cooled to 0° C.; 1 mL of NaOH (15 wt %) aqueous solution and 1 mL of water were added successively; the resulting mixture was filtered with diatomaceous earth; the filtrate was spin-dried to yield 680 mg of a white solid; the white solid is (4-(2-(piperidin-1-yl)ethoxy)phenyl)methanol (Compound 3) in 88.7% yield.

¹H NMR(CDCl₃, 300 MHz) δ: 7.30 (d, J=6.0 Hz, 2H), 6.92 (d, J=6.0 Hz, 2H), 4.64 (s, 2H), 4.17 (t, J=6.0 Hz, 2H), 2.98 (t, J=6.0 Hz, 2H), 2.74 (m, 4H), 1.89-1.86 (m, 6H)

### Step 3. Preparation of 1-(2-(4-(((4-nitronaphthalen-1-yl)oxy)methyl)phenoxy)ethyl)piperidine (Compound 4)

1.03 g of (4-(2-(piperidin-1-yl)ethoxy)phenyl)methanol (Compound 3) (4.39 mmol, 1.2 eq) was dissolved in 30 mL of anhydrous THF to form a solution; the solution was cooled to 0° C.; 293 mg of NaH (7.32 mmol, 2 eq) was added in batches and allowed to stand for 0.5 h; 700 mg of 1-fluoro-4-nitronaphthalene (3.66 mmol, 1.0 eq) was added and allowed to react at room temperature for 12 h; when the TLC plate showed that no more starting materials were left in the reaction time, 100 mL of saturated ammonium chloride aqueous solution was added to form a resulting mixture; the resulting mixture was extracted three times with ethyl acetate (each time 100 mL); the organic phases were mixed together; the mixed organic phase was dried with anhydrous sodium sulfate, spin-dried, and passes through the spin column (a ratio of the volume of dichloromethane to methanol is (60:1)-(20:1)) to yield 710 mg of a yellow solid; the yellow solid is 1-(2-(4-(((4-nitronaphthalen-1-yl)oxy) methyl)phenoxy)ethyl)piperidine (Compound 4) in 47.6% yield.

¹H NMR (CDCl₃, 300 MHz) 8.20 (d, J=9.0 Hz, 1H), 8.13 (d, J=9.0 Hz, 2H), 7.63-7.52 (m, 2H), 7.34-7.21 (m, 3H), 6.92 (d, J=9.0 Hz, 1H), 6.82 (d, J=9.0 Hz, 1H), 4.50 (s, 2H), 4.37 (t, J=6.0 Hz, 2H), 3.55-3.30 (m, 4H), 2.97 (t, J=6.0 Hz, 2H), 1.79-1.67 (m, 4H), 1.65 (m, 4H), 1.39-1.20 (m, 2H).

### Step 4 4-((4-(2-(piperidin-1-yl)ethoxy)benzyl)oxy)naphthalen-1-amine (5)

700 mg of 1-(2-(4-(((4-nitronaphthalen-1-yl)oxy)methyl)phenoxy)ethyl)piperidine (Compound 4, 1.72 mmol, 1.0 eq) was dissolved in a mixed solution containing 25 mL of 1,2-dichloroethane and 25 mL of saturated ammonium chloride aqueous solution to yield a mixture; the mixture temperature was raised to 45° C.; 480 mg of iron powders (8.61 mmol, 5.0 eq) was slowly added in batches to the mixture; the temperature of the resulting mixture was raised to 55° C. and allowed to react for 2 h; when the TLC plate showed that no more starting materials were left in the reaction time, the product was filtered with diatomaceous earth; the filtrate was extracted three times with ethyl acetate (each time 100 mL); the organic phases was mixed together, dried with anhydrous sodium sulfate, spin-dried, and passes the elute through the spin column (a ratio of the volume of dichloromethane to methanol is (60:1)-(20:1)) to yield 350 mg of a yellow solid; the yellow solid is 4-((4-(2-(piperidin-1-yl)ethoxy)benzyl)oxy)naphthalen-1-amine (Compound 5) in 54% yield.

1H NMR (CDCl3, 300 MHz) 8.20 (d, J=9.0 Hz, 1H), 8.13 (d, J=9.0 Hz, 2H), 7.63-7.52 (m, 2H), 7.34-7.21 (m, 3H), 6.92 (d, J=9.0 Hz, 1H), 6.82 (d, J=9.0 Hz, 1H), 4.50 (s, 2H), 4.37 (t, J=6.0 Hz, 2H), 3.55-3.30 (m, 4H), 2.97 (t, J=6.0 Hz, 2H), 1.79-1.67 (m, 4H), 1.65 (m, 4H), 1.39-1.20 (m, 2H).

### Step 5. Preparation of 1-benzyl-3-(4-((4-(2-(piperidin-1-yl)ethoxy)benzyl)oxy)naphthalen-1-yl)urea (ID1120B-1)

200 mg of 4-((4-(2-(piperidin-1-yl)ethoxy)benzyl)oxy)naphthalen-1-amine (Compound 5, 0.53 mmol, 1.0 eq), 84.9 mg of benzyl isocyanate (0.64 mmol, 1.2 eq) and 137 mg of N,N-diisopropylethylamine (DIEA, 1.06 mmol, 2.0 eq) were dissolved in 25 mL of 1,2-dichloroethane and allowed to react at 85° C. for 12 h; when the TLC plate showed that no more starting materials are left in the reaction time, the resulting product was spin-dried and passes through the spin column (a ratio of the volume of dichloromethane to methanol is (50:1)-(15:1))) to yield 210 mg of a brown solid in 77.8% yield; the brown solid is 1-benzyl-3-(4-((4-(2-(piperidin-1-yl)ethoxy)benzyl)oxy)naphthalen-1-yl)urea (ID1120B-1).

¹H NMR(DMSO-d6, 300 MHz) δ: 8.32 (s, 1H), 8.19 (d, J=8.0 Hz, 1H), 8.01 (d, J=8.0 Hz, 2H), 7.68 (d, J=8.0 Hz, 2H), 7.58-7.26 (m, 8H), 7.05-6.98 (m, 3H), 6.82 (m, 1H), 5.20 (s, 2H), 4.34 (d, J=4.0 Hz, 2H), 4.11 (m, 2H), 2.52 (m, 2H), 1.53 (m, 4H), 1.40 (m, 2H), 1.39-1.20 (m, 2H).

The other compounds are synthesized according to the above method in Example 1, except for the following differences: the compound 1 is replaced in Step 1 and benzyl isocyanate is replaced in Step 5.

The compound ID1120B-P is named 1-benzyl-3-(4-((4-(2-(piperidin-1-yl)ethoxy)benzyl)oxy)naphthalen-1-yl)urea phosphate.

The compound ID1120B-P is synthesized by the following route:

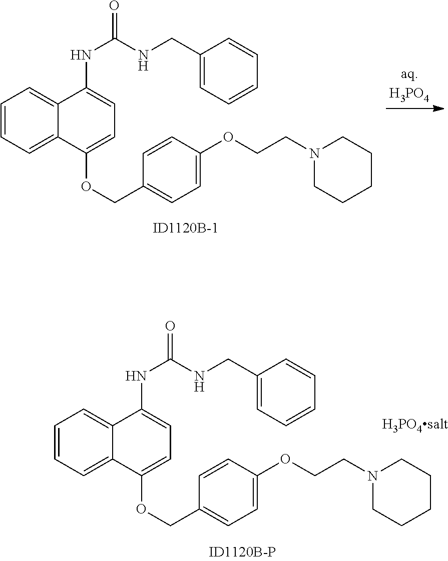 [SMILES: O=C(NCc1ccccc1)Nc1ccc(OCc2ccc(OCCN3CCCCC3)cc2)c2ccccc12 ; O=C(NCc1ccccc1)Nc1ccc(OCc2ccc(OCCN3CCCCC3)cc2)c2ccccc12]

2100 mg of ID1120B-1 (0.20 mmol, 1.0 eq) was dissolved in 10 mL of dimethyl sulfoxide (DMSO) to yield a mixture; 45 mg of 85% phosphoric acid (0.40 mmol, 2.0 eq) was add to the mixture and allowed to react at 50° C. for 2 h; when the TLC plate showed that no more starting materials were left in the reaction time, 50 mL of water was added to the resulting product; the resulting product was extracted twice with dichloromethane and methanol (a volume ratio of dichloromethane to methanol was 10:1); the organic phases was mixed together and dried with anhydrous sodium sulfate to yield 115 mg of a brown solid in 90% yield; the brown solid is 1-benzyl-3-(4-((4-(2-(piperidin-1-yl)ethoxy)benzyl)oxy)naphthalen-1-yl)urea phosphate (ID1120B-P).

The NMR parameters of other compounds are as follows:

ID1120C-1 ¹H NMR(DMSO-d6, 300 MHz) δ: 8.35 (s, 1H), 8.18 (d, J=8.0 Hz, 1H), 8.02 (d, J=8.0 Hz, 2H), 7.69 (d, J=8.0 Hz, 2H), 7.58-7.26 (m, 7H), 7.05-6.98 (m, 3H), 6.82 (m, 1H), 5.20 (s, 2H), 4.34 (d, J=4.0 Hz, 2H), 4.11 (m, 2H), 2.52 (m, 2H), 1.53 (m, 4H), 1.40 (m, 2H), 1.39-1.20 (m, 2H).

ID1120D-1 ¹H NMR(DMSO-d6, 300 MHz) δ: 8.33 (s, 1H), 8.20 (d, J=8.0 Hz, 1H), 8.03 (d, J=8.0 Hz, 2H), 7.71 (d, J=8.0 Hz, 2H), 7.58-7.26 (m, 7H), 7.05-6.98 (m, 3H), 6.82 (m, 1H), 5.20 (s, 2H), 4.34 (d, J=4.0 Hz, 2H), 4.11 (m, 2H), 2.52 (m, 2H), 1.53 (m, 4H), 1.40 (m, 2H), 1.39-1.20 (m, 2H).

IY210119B-1 ¹H NMR(DMSO-d6, 300 MHz) δ: 8.55 (s, 1H), 8.19 (d, J=6.0 Hz, 1H), 8.09 (d, J=6.0 Hz, 1H), 7.67 (d, J=6.0 Hz, 1H), 7.57-7.36 (m, 6H), 7.20-7.12 (m, 2H), 7.06-7.02 (m, 6H), 5.23 (s, 2H), 4.42-4.40 (m, 2H), 4.32 (d, J=3.0 Hz, 2H), 3.62-3.60 (m, 4H), 3.13-3.11 (m, 2H), 1.80-1.72 (m, 4H), 1.27-1.23 (m, 2H).

IY210115B-1 ¹H NMR(DMSO-d6, 300 MHz) δ: 8.59 (s, 1H), 8.19 (d, J=6.0 Hz, 1H), 8.08 (d, J=6.0 Hz, 1H), 7.63 (d, J=6.0 Hz, 1H), 7.52-7.50 (m, 4H), 7.17-7.03 (m, 7H), 5.23 (s, 2H), 4.36-4.34 (m, 4H), 3.62-3.60 (m, 4H), 3.13-3.11 (m, 2H), 1.80-1.72 (m, 4H), 1.27-1.23 (m, 2H).

IY210118B-1 ¹H NMR(DMSO-d6, 300 MHz) δ: 8.65 (s, 1H), 8.21 (d, J=6.0 Hz, 1H), 8.12 (d, J=6.0 Hz, 1H), 7.70 (d, J=6.0 Hz, 1H), 7.58-7.34 (m, 6H), 7.20-7.12 (m, 2H), 7.06-7.02 (m, 6H), 5.23 (s, 2H), 4.42-4.40 (m, 2H), 4.32 (d, J=3.0 Hz, 2H), 3.62-3.60 (m, 4H), 3.13-3.11 (m, 2H), 1.80-1.72 (m, 4H), 1.27-1.23 (m, 2H).

IY210113D-1 ¹H NMR(DMSO-d6, 300 MHz) δ: 8.57 (s, 1H), 8.19 (d, J=6.0 Hz, 1H), 8.08 (d, J=6.0 Hz, 1H), 7.71 (d, J=6.0 Hz, 1H), 7.56-7.51 (m, 6H), 7.17 (m, 1H), 7.04-7.02 (m, 3H), 5.22 (s, 2H), 4.43-4.41 (m, 4H), 3.62-3.60 (m, 4H), 3.13-3.11 (m, 2H), 1.80-1.72 (m, 4H), 1.27-1.23 (m, 2H).

IY1210B-1 ¹H NMR(DMSO-d6, 300 MHz) δ: 8.37 (s, 1H), 8.18 (d, J=6.0 Hz, 1H), 8.05 (d, J=6.0 Hz, 2H), 7.70 (d, J=6.0 Hz, 1H), 7.57-7.25 (m, 10H), 7.06-7.00 (m, 4H), 5.19 (s, 2H), 4.88-4.84 (m, 1H), 4.25 (t, J=3.0 Hz, 2H), 2.30 (t, J=3.0 Hz, 2H), 1.64-1.51 (m, 4H), 1.40-1.30 (m, 5H).

ID210106D-1 ¹H NMR(DMSO-d6, 300 MHz) δ: 8.47 (s, 1H), 8.19 (d, J=6.0 Hz, 1H), 8.04 (d, J=6.0 Hz, 1H), 7.64 (d, J=6.0 Hz, 1H), 7.58-7.51 (m, 4H), 7.38-7.29 (m, 4H), 7.06-7.03 (m, 4H), 5.26 (s, 2H), 4.39-4.33 (m, 4H), 3.49-3.43 (m, 4H), 3.01-2.99 (m, 2H), 1.80-1.72 (m, 4H), 1.27-1.23 (m, 2H).

ID210118D-1 ¹H NMR(DMSO-d6, 300 MHz) δ: 8.62 (s, 1H), 8.19 (d, J=6.0 Hz, 1H), 8.11 (d, J=6.0 Hz, 1H), 7.68 (d, J=6.0 Hz, 1H), 7.56-7.24 (m, 9H), 7.05-7.02 (m, 3H), 5.22 (s, 2H), 4.42-4.40 (m, 2H), 4.34 (d, J=3.0 Hz, 2H), 3.62-3.60 (m, 4H), 3.13-3.11 (m, 2H), 1.80-1.72 (m, 4H), 1.27-1.23 (m, 2H).

ID210113C-1 ¹H NMR(DMSO-d6, 300 MHz) δ: 8.41 (s, 1H), 8.19 (d, J=6.0 Hz, 1H), 8.04 (d, J=6.0 Hz, 1H), 7.68 (d, J=6.0 Hz, 1H), 7.58-7.49 (m, 4H), 7.27-7.25 (m, 2H), 7.06-7.02 (m, 3H), 6.93-6.90 (m, 3H), 5.23 (s, 2H), 4.41 (t, J=3.0 Hz, 2H), 4.26 (d, J=6.0 Hz, 2H), 3.74 (s, 3H), 3.52-3.49 (m, 4H), 3.13-3.11 (m, 2H), 1.80-1.72 (m, 4H), 1.27-1.23 (m, 2H).

IY210103C-1 ¹H NMR(DMSO-d6, 300 MHz) δ: 8.23-8.21 (m, 2H), 8.08 (d, J=6.0 Hz, 1H), 7.74 (d, J=6.0 Hz, 1H), 7.64-7.44 (m, 5H), 7.40-7.37 (m, 8H), 7.23-7.21 (m, 1H), 7.08-7.04 (m, 3H), 5.25 (s, 2H), 4.38 (m, 2H), 3.49-3.43 (m, 4H), 3.13-3.01 (m, 2H), 1.80-1.72 (m, 4H), 1.27-1.23 (m, 2H).

ID210118C-1 ¹H NMR(DMSO-d6, 300 MHz) δ: 8.23 (m, 2H), 8.09 (d, J=6.0 Hz, 1H), 7.70 (d, J=6.0 Hz, 1H), 7.54-7.47 (m, 5H), 7.42-7.39 (m, 7H), 7.23-7.21 (m, 1H), 7.10-7.06 (m, 3H), 5.25 (s, 2H), 4.38 (m, 2H), 3.49-3.43 (m, 4H), 3.13-3.01 (m, 2H), 1.80-1.72 (m, 4H), 1.27-1.23 (m, 2H).

ID210115B-1 ¹H NMR(DMSO-d6, 300 MHz) δ: 8.62 (s, 1H), 8.19 (d, J=6.0 Hz, 1H), 8.09 (d, J=6.0 Hz, 1H), 7.65 (d, J=6.0 Hz, 1H), 7.56-7.35 (m, 8H), 7.05-7.02 (m, 3H), 5.22 (s, 2H), 4.39-4.30 (m, 4H), 3.62-3.60 (m, 4H), 3.13-3.11 (m, 2H), 1.80-1.72 (m, 4H), 1.27-1.23 (m, 2H).

ID210114B-1 ¹H NMR(DMSO-d6, 300 MHz) δ: 8.68 (s, 1H), 8.19 (d, J=6.0 Hz, 1H), 8.08 (d, J=6.0 Hz, 1H), 7.81-7.50 (m, 8H), 7.30 (brs, 1H), 7.05-7.02 (m, 3H), 5.22 (s, 2H), 4.40-4.38 (m, 4H), 3.62-3.60 (m, 4H), 3.13-3.11 (m, 2H), 1.80-1.72 (m, 4H), 1.27-1.23 (m, 2H).

ID1210B-1 ¹H NMR(DMSO-d6, 300 MHz) δ: 8.34 (s, 1H), 8.18 (d, J=6.0 Hz, 1H), 8.00 (d, J=6.0 Hz, 2H), 7.50 (d, J=6.0 Hz, 1H), 7.33-7.26 (m, 8H), 7.04-7.01 (m, 3H), 6.47 (t, J=6.0 Hz, 1H), 5.22 (s, 2H), 4.34 (m, 2H), 3.58 (t, J=3.0 Hz, 2H), 2.30 (t, J=3.0 Hz, 2H), 1.53-1.51 (m, 4H), 1.40-1.39 (m, 2H).

IY1207A-1 ¹H NMR(CDCl3, 300 MHz) δ: 8.26 (d, J=6.0 Hz, 1H), 7.66 (d, J=6.0 Hz, 1H), 8.19 (d, J=6.0 Hz, 1H), 7.46 (s, 1H), 7.34-7.07 (m, 10H), 6.87 (d, J=6.0 Hz, 2H), 6.77 (d, J=6.0 Hz, 1H), 5.83 (brs, 1H), 5.09 (s, 2H), 4.75 (d, J=3.0 Hz, 2H), 4.13 (t, J=3.0 Hz, 2H), 2.81 (t, J=3.0 Hz, 2H), 2.56 (m, 4H), 1.61 (t, J=6.0 Hz, 4H), 1.40-1.39 (m, 2H).

IY1223B-1 ¹H NMR(DMSO-d6, 300 MHz) δ: 8.56 (s, 1H), 8.22 (d, J=6.0 Hz, 1H), 8.07 (d, J=6.0 Hz, 2H), 7.71 (d, J=6.0 Hz, 1H),7.61-7.48 (m, 4H), 7.29 (t, J=6.0 Hz, 2H), 7.09-6.96 (m, 3H), 5.25 (s, 2H), 4.41 (m, 2H), 3.50-3.45 (m, 2H), 3.02 (m, 2H), 1.68-1.27 (m, 6H).

IY1214A-1 ¹H NMR(DMSO-d6, 300 MHz) δ: 8.69 (s, 1H), 8.26 (d, J=6.0 Hz, 1H), 8.19 (d, J=6.0 Hz, 2H), 7.76 (d, J=6.0 Hz, 1H),7.59-7.56 (m, 4H), 7.45 (d, J=6.0 Hz, 2H), 7.13-7.09 (m, 3H), 6.92 (d, J=6.0 Hz, 2H), 5.30 (s, 2H), 4.45 (m, 2H), 3.55-3.54 (m, 4H), 3.17 (t, J=3.0 Hz, 2H), 1.84-1.80 (m, 4H), 1.32-1.28 (m, 2H).

ID1214B-1 ¹H NMR(DMSO-d6, 300 MHz) δ: 9.75 (brs, 1H), 8.85 (brs, 1H), 8.22 (d, J=6.0 Hz, 1H), 8.11 (d, J=6.0 Hz, 2H), 8.10 (s, 1H),7.69-7.56 (m, 5H), 7.38-7.35 (m, 1H), 7.07 (t, J=3.0 Hz, 3H), 5.26 (s, 2H), 4.38 (m, 2H), 3.61-3.50 (m, 4H), 3.00 (t, J=3.0 Hz, 2H), 1.80-1.71 (m, 4H), 1.29-1.26 (m, 2H).

IY1225B-1 ¹H NMR(DMSO-d6, 300 MHz) δ: 8.72 (s, 1H),8.20 (d, J=6.0 Hz, 1H), 8.10 (d, J=6.0 Hz, 2H), 7.71 (d, J=6.0 Hz, 1H), 7.60-7.53 (m, 4H), 7.31-7.29 (m, 1H), 7.08 (t, J=6.0 Hz, 3H), 5.25 (s, 2H), 4.40 (m, 2H), 3.81 (s, 3H), 3.51-3.45 (m, 4H), 3.02 (m, 2H), 1.80-1.72 (m, 4H), 1.39-1.27 (m, 2H).

IY1210A-1 ¹H NMR(DMSO-d6, 300 MHz) δ: 9.98 (brs, 1H), 8.95 (m, 1H), 8.23-8.06 (m, 2H), 7.70-7.49 (m, 4H), 7.09-6.99 (m, 2H), 7.85-6.59 (m, 1H), 5.20 (s, 2H), 4.33 (d, J=3.0 Hz, 2H), 4.11 (t, J=3.0 Hz, 2H), 2.30 (t, J=3.0 Hz, 2H), 1.53-1.51 (m, 4H), 1.40-1.39 (m, 2H).

IY1226B-1 ¹H NMR(DMSO-d6, 300 MHz) δ: 8.80 (s, 1H), 8.22 (d, J=6.0 Hz, 1H), 8.13 (d, J=6.0 Hz, 2H), 7.70 (d, J=6.0 Hz, 1H), 7.61-7.47 (m, 4H), 7.30-7.28 (m, 1H), 7.08-7.01 (m, 4H), 5.26 (s, 2H), 4.39 (m, 2H), 3.82 (s, 3H), 3.50-3.45 (m, 4H), 3.02 (m, 2H), 1.81-1.72 (m, 4H), 1.40-1.27 (m, 2H).

IY1229C-1 ¹H NMR(DMSO-d6, 300 MHz) δ: 8.22-8.17 (m, 2H), 7.74-7.71 (m, 2H), 7.69-7.44 (m, 5H), 7.08-7.04 (m, 4H), 5.25 (s, 2H), 4.41 (m, 2H), 3.48-3.39 (m, 4H), 3.14-3.12 (m, 2H), 1.79-1.71 (m, 4H), 1.27-1.23 (m, 2H).

ID1229C-1 ¹H NMR(DMSO-d6, 300 MHz) δ: 8.21 (d, J=6.0 Hz, 1H), 8.13 (d, J=6.0 Hz, 2H), 7.93 (s, 1H), 7.68 (d, J=6.0 Hz, 1H), 7.69-7.43 (m, 5H), 7.08-7.04 (m, 3H), 5.25 (s, 2H), 4.39 (m, 2H), 3.48-3.39 (m, 4H), 3.14-3.12 (m, 2H), 1.79-1.71 (m, 4H), 1.27-1.23 (m, 2H).

ID1229D-1 ¹H NMR(DMSO-d6, 300 MHz) δ: 8.21 (d, J=6.0 Hz, 1H), 8.07 (s, 1H), 7.53 (d, J=6.0 Hz, 2H), 7.48-7.47 (m, 6H), 7.09-7.04 (m, 3H), 5.26 (s, 2H), 4.42 (m, 2H), 3.61-3.59 (m, 4H), 3.40 (s, 3H), 3.12-3.11 (m, 2H), 1.79-1.60 (m, 6H).

ID1224D-1 ¹H NMR(DMSO-d6, 300 MHz) δ: 8.74 (s, 1H), 8.22 (d, J=6.0 Hz, 1H), 8.11 (d, J=6.0 Hz, 2H), 7.70 (d, J=6.0 Hz, 1H), 7.61-7.52 (m, 4H), 7.30-7.28 (m, 1H), 7.07 (t, J=6.0 Hz, 3H), 5.26 (s, 2H), 4.39 (m, 2H), 3.82 (s, 3H), 3.50-3.45 (m, 4H), 3.02 (m, 2H), 1.81-1.72 (m, 4H), 1.40-1.27 (m, 2H).

ID1231B-1 ¹H NMR(DMSO-d6, 300 MHz) δ: 8.80 (s, 1H), 8.21 (d, J=6.0 Hz, 1H), 8.10 (d, J=6.0 Hz, 2H), 7.39 (d, J=6.0 Hz, 1H), 7.51-7.48 (m, 4H), 7.32-7.29 (m, 1H), 7.07 (t, J=6.0 Hz, 3H), 5.22 (s, 2H), 4.40 (m, 2H), 3.81 (s, 3H), 3.51-3.46 (m, 4H), 3.01 (m, 2H), 1.81-1.72 (m, 4H), 1.40-1.27 (m, 2H).

IY1214B-2 ¹H NMR(DMSO-d6, 300 MHz) δ: 8.75 (s, 1H), 8.20 (d, J=6.0 Hz, 1H), 8.15 (d, J=6.0 Hz, 2H), 7.41 (d, J=6.0 Hz, 1H), 7.35-7.30 (m, 5H), 7.30-7.28 (m, 1H), 7.02 (t, J=6.0 Hz, 3H), 5.23 (s, 2H), 4.38 (m, 2H), 3.80 (s, 3H), 3.51-3.46 (m, 4H), 3.01 (m, 2H), 1.81-1.72 (m, 4H), 1.40-1.27 (m, 2H).

ID1224C-1 ¹-H NMR(DMSO-d6, 300 MHz) δ: 8.59 (s, 1H), 8.21 (d, J=6.0 Hz, 1H), 8.11 (d, J=6.0 Hz, 2H), 7.71 (d, J=6.0 Hz, 1H), 7.60-7.50 (m, 4H), 7.26-7.25 (m, 1H), 7.07-7.02 (m, 3H), 6.91-6.88 (m, 2H), 5.24 (s, 2H), 4.30 (m, 2H), 3.74 (s, 3H), 3.71 (s, 3H), 3.60 (m, 2H), 3.14 (m, 2H), 1.68-1.18 (m, 6H).

IY1229D-1 ¹H NMR(DMSO-d6, 300 MHz) δ: 8.50 (s, 1H), 8.19 (d, J=6.0 Hz, 1H), 8.05 (d, J=6.0 Hz, 1H), 7.51 (d, J=6.0 Hz, 1H), 7.40-7.37 (m, 8H), 7.04-7.00 (m, 4H), 5.23 (s, 2H), 4.39 (m, 2H), 3.48-3.39 (m, 4H), 3.14-3.12 (m, 2H), 1.79-1.71 (m, 4H), 1.27-1.23 (m, 2H).

IY210103B-1 ¹H NMR(DMSO-d6, 300 MHz) δ: 8.84 (brs, 1H), 8.21 (d, J=6.0 Hz, 1H), 8.13 (d, J=6.0 Hz, 1H), 7.84 (d, J=6.0 Hz, 1H), 7.69-7.51 (m, 5H), 7.34-7.33 (m, 2H), 7.08-7.04 (m, 3H), 5.25 (s, 2H), 4.39 (m, 2H), 3.48-3.43 (m, 4H), 3.01-2.99 (m, 2H), 1.79-1.72 (m, 4H), 1.27-1.23 (m, 2H).

IY210105B-1 ¹H NMR(DMSO-d6, 300 MHz) δ: 8.70 (brs, 1H), 8.21 (d, J=6.0 Hz, 1H), 8.12 (d, J=6.0 Hz, 1H), 7.70 (d, J=6.0 Hz, 1H), 7.54-7.52 (m, 5H), 7.13-6.94 (m, 5H), 5.26 (s, 2H), 4.39 (m, 2H), 3.81 (s, 3H), 3.49-3.43 (m, 4H), 3.01-2.99 (m, 2H), 1.80-1.72 (m, 4H), 1.27-1.23 (m, 2H).

IY210105C-1 ¹H NMR(DMSO-d6, 300 MHz) δ: 8.83 (s, 1H), 8.22 (d, J=6.0 Hz, 1H), 8.10 (d, J=6.0 Hz, 1H), 7.69 (d, J=6.0 Hz, 1H), 7.60-7.55 (m, 11H), 5.25 (s, 2H), 4.40 (t, J=6.0 Hz, 2H), 3.82 (s, 3H), 3.49-3.43 (m, 4H), 3.01-2.99 (m, 2H), 1.80-1.72 (m, 4H), 1.27-1.23 (m, 2H).

ID210105C-1 ¹H NMR(DMSO-d6, 300 MHz) δ: 8.80 (s, 1H), 8.21 (d, J=6.0 Hz, 1H), 8.11 (d, J=6.0 Hz, 1H), 7.70 (d, J=6.0 Hz, 1H), 7.74-7.05 (m, 11H), 5.26 (s, 2H), 4.40 (t, J=6.0 Hz, 2H), 3.49-3.43 (m, 4H), 3.01-2.99 (m, 2H), 1.80-1.72 (m, 4H), 1.27-1.23 (m, 2H).

IY210105D-1 ¹H NMR(DMSO-d6, 300 MHz) δ: 8.78 (brs, 1H), 8.25 (d, J=6.0 Hz, 1H), 8.17 (d, J=6.0 Hz, 1H), 7.75 (d, J=6.0 Hz, 1H), 7.50-7.49 (m, 5H), 7.10-6.96 (m, 5H), 5.25 (s, 2H), 4.41 (m, 2H), 3.40-3.38 (m, 4H), 3.01-2.99 (m, 2H), 1.81-1.72 (m, 4H), 1.27-1.23 (m, 2H).

IY210105A-1 ¹H NMR(DMSO-d6, 300 MHz) δ: 8.76 (brs, 1H), 8.23 (d, J=6.0 Hz, 1H), 8.16 (d, J=6.0 Hz, 1H), 7.73 (d, J=6.0 Hz, 1H), 7.51-7.49 (m, 4H), 7.10-6.92 (m, 5H), 5.23 (s, 2H), 4.37 (m, 2H), 3.45-3.43 (m, 4H), 3.01-2.99 (m, 2H), 1.84-1.72 (m, 4H), 1.27-1.23 (m, 2H).

IY210106D-1 ¹H NMR(DMSO-d6, 300 MHz) δ: 8.62 (brs, 1H), 8.19 (d, J=6.0 Hz, 1H), 8.11 (d, J=6.0 Hz, 1H), 7.69 (d, J=6.0 Hz, 1H), 7.52-7.49 (m, 4H), 7.11-6.94 (m, 5H), 5.22 (s, 2H), 4.39 (m, 2H), 3.49-3.43 (m, 4H), 3.01-2.99 (m, 2H), 1.80-1.72 (m, 4H), 1.27-1.23 (m, 2H).

ID210110C-1 ¹H NMR(DMSO-d6, 300 MHz) δ: 8.60 (s, 1H), 8.18 (d, J=6.0 Hz, 1H), 8.13 (d, J=6.0 Hz, 1H), 7.71 (d, J=6.0 Hz, 1H), 7.55-7.51 (m, 4H), 7.11-6.94 (m, 5H), 5.22 (s, 2H), 4.39 (m, 2H), 3.81 (s, 3H), 3.49-3.43 (m, 4H), 3.01-2.99 (m, 2H), 1.80-1.72 (m, 4H), 1.27-1.23 (m, 2H).

IY210110D-1 ¹H NMR(DMSO-d6, 300 MHz) δ: 8.58 (s, 1H), 8.16 (d, J=6.0 Hz, 1H), 8.11 (d, J=6.0 Hz, 1H), 7.69 (d, J=6.0 Hz, 1H), 7.55-7.51 (m, 4H), 7.11-6.94 (m, 5H), 5.22 (s, 2H), 4.39 (m, 2H), 3.81 (s, 3H), 3.49-3.43 (m, 4H), 3.01-2.99 (m, 2H), 1.80-1.72 (m, 4H), 1.27-1.23 (m, 2H).

ID1207B-1 ¹H NMR(DMSO-d6, 300 MHz) δ: 8.18 (d, J=6.0 Hz, 1H), 8.10 (s, 1H), 8.01 (d, J=6.0 Hz, 2H), 7.71 (d, J=6.0 Hz, 1H),7.59-7.46 (m, 4H), 7.03-6.99 (m, 3H), 6.42(d, J=3.0 Hz, 1H), 5.19 (s, 2H), 4.00 (m, 2H), 3.99-3.95 (m, 1H), 2.33-2.30 (m, 2H), 1.87-1.84 (m, 2H), 1.68-1.40 (m, 8H), 1.27-1.24 (m, 2H).

ID1217B-1 ¹H NMR(DMSO-d6, 300 MHz) δ: 8.20 (d, J=6.0 Hz, 1H), 8.11 (s, 1H), 8.05 (d, J=6.0 Hz, 2H), 7.70 (d, J=6.0 Hz, 1H), 7.56-7.48 (m, 4H), 7.05-6.98 (m, 3H), 6.48(d, J=3.0 Hz, 1H), 5.20 (s, 2H), 4.00 (m, 2H), 3.99-3.97 (m, 1H), 2.34-2.32 (m, 2H), 1.85-1.84 (m, 2H), 1.68-1.40 (m, 10H), 1.27-1.24 (m, 2H).

ID1223A-1 ¹H NMR(DMSO-d6, 300 MHz) δ: 8.28 (s, 1H), 8.18 (d, J=6.0 Hz, 1H), 8.00 (d, J=6.0 Hz, 2H), 7.64 (d, J=6.0 Hz, 2H), 7.45-7.47 (m, 4H), 7.03-6.98 (m, 3H), 5.93 (s, 2H), 5.19 (s, 2H), 4.11 (t, J=3.0 Hz, 2H), 2.74 (t, J=3.0 Hz, 2H), 1.53-1.51 (m, 4H), 1.41-1.39 (m, 2H).

ID1215B-1 ¹H NMR(DMSO-d6, 300 MHz) δ: 8.20 (s, 1H), 8.18 (d, J=6.0 Hz, 1H), 8.00 (d, J=6.0 Hz, 2H), 7.66 (d, J=6.0 Hz, 1H), 7.48-7.46 (m, 3H), 7.03-6.99 (m, 3H), 6.38 (t, J=3.0 Hz, 1H), 5.20 (s, 2H), 4.12 (m, 2H), 2.70-2.68 (m, 4H), 1.54-1.27 (m, 8H).

ID1215C-1 ¹H NMR(DMSO-d6, 300 MHz) δ: 8.22 (s, 1H), 8.18 (d, J=6.0 Hz, 1H), 8.01 (d, J=6.0 Hz, 1H), 7.69 (d, J=6.0 Hz, 1H), 7.51-7.48 (m, 3H), 7.03-7.01 (m, 3H), 6.53-6.51 (m, 1H), 5.21 (s, 2H), 4.42-4.40 (m, 2H), 3.86-3.83 (m, 2H), 3.70-3.68 (m, 2H), 3.41-3.38 (m, 4H), 1.85-1.24 (m, 13H).

IY1215C-1 ¹H NMR(DMSO-d6, 300 MHz) δ: 8.19 (d, J=6.0 Hz, 1H), 8.13 (s, 1H), 8.00 (d, J=6.0 Hz, 2H), 7.72 (d, J=6.0 Hz, 1H), 7.56-7.44 (m, 3H), 7.01-6.99 (m, 3H), 6.40 (d, J=3.0 Hz, 1H), 5.19 (s, 2H), 4.01 (m, 2H), 4.00-3.95 (m, 1H), 2.30-2.28 (m, 2H), 1.86-1.84 (m, 2H), 1.67-1.40 (m, 10H), 1.27-1.24 (m, 2H).

ID1215A-1 ¹H NMR(DMSO-d6, 300 MHz) δ: 8.18 (s, 1H), 8.10 (d, J=6.0 Hz, 1H), 8.02 (d, J=6.0 Hz, 1H), 7.70 (d, J=6.0 Hz, 1H), 7.50-7.48 (m, 3H), 7.02-7.01 (m, 3H), 6.53-6.51 (m, 1H), 5.20 (s, 2H), 4.43-4.40 (m, 2H), 3.85-3.83 (m, 2H), 3.69-3.68 (m, 2H), 3.41-3.38 (m, 4H), 1.85-1.24 (m, 13H).

IY1215D-1 ¹H NMR(DMSO-d6, 300 MHz) δ: 8.24 (s, 1H), 8.19 (d, J=6.0 Hz, 1H), 8.03 (d, J=6.0 Hz, 1H), 7.68 (d, J=6.0 Hz, 1H), 7.50-7.48 (m, 3H), 7.04-7.01 (m, 3H), 6.52-6.50 (m, 1H), 5.20 (s, 2H), 4.41-4.39 (m, 2H), 3.85-3.83 (m, 2H), 3.72-3.68 (m, 2H), 3.40-3.37 (m, 4H), 1.84-1.24 (m, 13H).

IY210122C-1 ¹H NMR(DMSO-d6, 300 MHz) δ: 8.56 (brs, 1H), 8.18 (d, J=6.0 Hz, 1H), 8.11 (d, J=6.0 Hz, 1H), 7.69 (d, J=6.0 Hz, 1H), 7.52-7.50 (m, 5H), 7.14-7.06 (m, 5H), 5.26 (s, 2H), 4.40 (t, J=6.0 Hz, 2H), 3.49-3.43 (m, 4H), 3.01-2.99 (m, 2H), 1.80-1.72 (m, 4H), 1.27-1.23 (m, 2H).

ID210119B-1 ¹H NMR(DMSO-d6, 300 MHz) δ: 8.75 (brs, 1H), 8.21 (d, J=6.0 Hz, 1H), 8.14 (d, J=6.0 Hz, 1H), 7.70 (d, J=6.0 Hz, 1H), 7.54-7.51 (m, 5H), 7.15-7.05 (m, 5H), 5.26 (s, 2H), 4.40 (t, J=6.0 Hz, 2H), 3.49-3.43 (m, 4H), 3.01-2.99 (m, 2H), 1.80-1.72 (m, 4H), 1.27-1.23 (m, 2H).

ID210106C-1 ¹H NMR(DMSO-d6, 300 MHz) δ: 8.51 (s, 1H), 8.18 (d, J=6.0 Hz, 1H), 8.06 (d, J=6.0 Hz, 1H), 7.66 (d, J=6.0 Hz, 1H), 7.57-7.34 (m, 7H), 7.07-7.02 (m, 3H), 5.22 (s, 2H), 4.39-4.31 (m, 4H), 3.62-3.60 (m, 4H), 3.14-3.11 (m, 2H), 1.80-1.72 (m, 4H), 1.27-1.23 (m, 2H).

IY210128B-1 ¹H NMR(DMSO-d6, 300 MHz) δ: 8.75 (s, 1H), 8.22 (d, J=6.0 Hz, 1H), 8.14 (d, J=6.0 Hz, 1H), 7.74 (d, J=6.0 Hz, 1H), 7.65-7.32 (m, 10H), 7.09-7.05 (m, 3H), 5.26 (s, 2H), 4.39 (m, 2H), 3.49-3.45 (m, 4H), 3.02-3.00 (m, 2H), 1.80-1.71 (m, 4H), 1.29-1.24 (m, 2H).

ID210127B-1 ¹H NMR(DMSO-d6, 300 MHz) δ: 8.25 (s, 1H), 8.17 (d, J=6.0 Hz, 1H), 8.02 (d, J=6.0 Hz, 1H), 7.70 (d, J=6.0 Hz, 1H), 7.50-7.48 (m, 4H), 7.04-7.01 (m, 3H), 6.53-6.51 (m, 1H), 5.20 (s, 2H), 4.41-4.40 (m, 2H), 3.84-3.83 (m, 2H), 3.71-3.68 (m, 2H), 3.41-3.38 (m, 4H), 1.84-1.24 (m, 13H).

### Example 2

Uses of naphthylurea compound ID1120B-1 and a phosphate ID1120B-P thereof to inhibit proliferation of cancer cells in liver cancer, breast cancer, lung cancer, gefitinib- or afatinib-resistant lung cancer and leukemia.

Difference cell lines HepG2, SMMC-7721, HuH-7, MCF-7, MDA-MB-231, MDA-MB-468, PC9, PC9-AR, PC9-GR, Jurkat and Molt-13 were harvested during log phase; the number of the cells in an original cell suspension was counted; the original cell suspension was diluted to a density of 5×10⁴ cells/mL; for a 96-well plate, 100 uL of the cell suspension was transferred to each well; DMSO is used as solvent for negative control; (2E)-3-(6-bromo-2-pyridyl)-2-cyano-N-[(1S)-1-phenylethyl]-2-acrylamide (WP1066CAS: 857064-38-1, with a formula

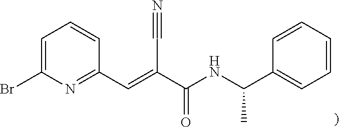 [SMILES: C[C@H](NC(=O)/C(C#N)=C/c1cccc(Br)n1)c1ccccc1]

or gefitinib was used as a positive control; the naphthylurea compound ID1120B-1 and the phosphate ID1120B-P thereof were diluted with DMSO and added into the 96-well plate to achieve a final concentration of 0.1, 0.3, 1, 3, 10, 30, 100 and 300 μmol/L in each well; the 96-well plate was incubated for 48 h; 10 μL of MTT solvent (5 mg/mL) was added into each well; the 96-well plate was incubated at 37° C. for 4 h; a culture supernatant was discarded; 150 μL of DMSO was added into each well; the 96-well plate was shaken for 10 min on a plate shaker; an optical density (OD) of the resulting product was measured at a wavelength of 490 nm by an ELISA reader. Test results were recorded. A cell growth curve was drawn with the dosage of each compound as abscissa and the absorbance value as ordinate. The half maximum inhibition rates (IC50 value) of the cancer cells were shown in Table 1, FIGS. 1A-1J and FIGS. 2A-2J.

TABLE 1
Half maximum inhibition rate (IC50 value) of cancer cells

| Cell line | Name | IC50 (μM) |
|---|---|---|
| HepG2 | ID1120B-1 | 1.677 |
| | IY210119B-1 | 3.505 |
| | IY210115B-1 | 5.769 |
| | IY210113D-1 | 3.363 |
| | IY1210B-1 | 1.953 |
| | ID210106D-1 | 5.253 |
| | ID210118D-1 | 36.98 |
| | ID210113C-1 | 1.312 |
| | IY210113C-1 | 3.936 |
| | ID210115B-1 | 4.649 |
| | ID210114B-1 | 3.969 |
| | ID1214B-1 | 0.8799 |
| | IY1225B-1 | 1.953 |
| | IY1210A-1 | 3.844 |
| | IY1226B-1 | 2.603 |
| | IY1229C-1 | 6.431 |
| | ID1229C-1 | 50.3 |
| | ID1229D-1 | 27.17 |
| | ID1224D-1 | 32.16 |
| | ID1231B-1 | 16 |
| | IY1214B-2 | 3.852 |
| | ID1224C-1 | 93.82 |
| | IY1229D-1 | 12.23 |
| | IY210103B-1 | 68.18 |
| | IY210105B-1 | 43.8 |
| | IY210105C-1 | 4.387 |
| | ID210105C-1 | 66.9 |
| | IY210105D-1 | 3.796 |
| | ID1207B-1 | 462.4 |
| | ID1217B-1 | 72.6 |
| | ID1223A-1 | 3.829 |
| | ID1215B-1 | 14.8 |
| | ID1215A-1 | 15.09 |
| | IY210122C-1 | 11 |
| | ID210119B-1 | 26.53 |
| | ID210127B-1 | 3.505 |
| | ID1120B-P | 2.37 |
| | Sorafenib | 6.172 |
| | WP1066 | 9.208 |
| | IY1214A-1 | 1.853 |
| SMMC-7721 | ID1120B-1 | 3.508 |
| | ID1120C-1 | 6.754 |
| | ID1120D-1 | 6.889 |
| | IY210119B-1 | 0.7357 |
| | IY210115B-1 | 40.12 |
| | IY210118B-1 | 23.64 |
| | ID1214B-1 | 8.735 |
| | WP1066 | 11.88 |
| | IY1214A-1 | 3.573 |
| | IY1214B-2 | 31.36 |
| | Sorafenib | 30.03 |
| HuH-7 | IY210113D-1 | 13.7 |
| | ID210106D-1 | 6.826 |
| | ID210118D-1 | 26.53 |
| | ID210113C-1 | 4.746 |
| | IY210113C-1 | 12.51 |
| | ID210118C-1 | — |
| | ID1120B-1 | 3.147 |
| | WP1066 | 8.108 |
| | IY1214A-1 | 2.757 |
| | IY1214B-2 | 23.1 |
| | Sorafenib | 17.55 |
| MCF-7 | ID210115B-1 | 39.02 |
| | ID210114B-1 | 13.18 |
| | ID1210B-1 | 8.083 |
| | IY1207A-1 | 24.3 |
| | IY1214A-1 | 3.541 |
| | ID1214B-1 | 88.44 |
| | WP1066 | 18.01 |
| MDA-MB-468 | IY1223B-1 | 13.18 |
| | IY1214A-1 | 39.02 |
| | ID1214B-1 | 688.5 |
| | IY1226B-1 | 99.15 |
| | ID1229C-1 | 47.47 |
| | ID1229D-1 | 524.6 |
| | ID1224D-1 | 9.054 |
| | ID1231B-1 | 24.3 |
| | IY1214B-2 | 3.852 |
| | IY1229D-1 | 14.25 |
| | IY210103B-1 | 20.36 |
| | IY210105B-1 | 51.35 |
| | IY210105C-1 | 1.885 |
| | IY210105D-1 | 3.796 |
| | IY210105A-1 | 20.36 |
| | IY210106D-1 | 6.345 |
| | ID210110C-1 | 6.129 |
| | IY20110D-1 | 0.6989 |
| | ID1207B-1 | 266.6 |
| | ID1217B-1 | 33.9 |
| | ID1223A-1 | 0.6472 |
| | ID1215B-1 | 2.216 |
| | ID1215C-1 | 3.467 |
| | IY1215C-1 | 5.359 |
| | ID1215A-1 | 2.168 |
| | IY210122C-1 | 11.64 |
| | ID210119B-1 | 26.55 |
| | IY210128B-1 | 62.5 |
| | ID210127B-1 | 0.7357 |
| | ID1214B-1 | 688.5 |
| | IY1214A-1 | 3.458 |
| | ID1120B-1 | 4.92 |
| | Gefitinib | 19.17 |
| | WP1066 | 13.22 |
| MDA-MB-231 | ID1120B-1 | 17.51 |
| | WP1066 | 10.15 |
| | IY1214A-1 | 2.106 |
| | ID1214B-1 | 113.7 |
| PC9 | ID1120B-1 | 14.07 |
| | WP1066 | 9.257 |
| | Gefitinib | 3.831 |
| PC9GR | ID1120B-1 | 32.32 |
| | WP1066 | 14.73 |
| | Gefitinib | 11.81 |
| PC9AR | ID1120B-1 | 8.004 |
| | WP1066 | 8.488 |
| | Gefitinib | 19.17 |
| Jurkat | ID1120B-1 | 3.231 |
| | ID1120B-P | 4.736 |
| | WP1066 | 10.03 |
| MOLT-13 | ID1120B-1 | 10.8 |
| | ID1120B-P | 9.785 |
| | WP1066 | 17.05 |

As shown in Table 1, the naphthylurea compound ID1120B-1 and the derivatives thereof, such as ID1214B-1, IY1214A-1 and IY1214B-2 are found to effectively inhibit the proliferation of the tumor cells in liver cancer, breast cancer, lung cancer and leukemia, especially in lung cancer.

### Example 3

Induction of cell cycle arrest at G2/M cycle in hepatoma cells by a compound ID1120B-1 and its derivatives ID1214B-1, IY1214A-1 and IY1214B-2.

HepG2 cells were harvested during log phase, digested, centrifuged and prepared into a single cell suspension; the number of the cells in the single cell suspension was counted; the cells were seeded into a 12-well plate, with 2×10⁵ cells per well; three wells were used as a parallel control design; 16 hours after seeding, the cells were treated with the compounds in a concentration gradient for 48 h; the cells were digested with trypsin and resuspended; the number of the cells in the cell suspension was counted and diluted to 5×10⁵ cells/mL; after the digestion was completed, the cell suspension was centrifuged; the supernatant was discarded; the pellet was washed twice with PBS (each time the mixture was centrifuged 2000 rpm for 5 min); the supernatant was discarded; a fixative comprising 980 μL of 70% cold ethanol and 20 μL of 5% BSA (a small amount of BSA reduces cellular stress and damage) was added to each microcentrifuge tube, so that the cells were fixed overnight at 4° C.; the fixative is discarded; the cells were washed three times in PBS to remove residual fixative (each time the mixture was centrifuged at 1000 rpm for 3 min); a DNA quantification kit is used to measure the content of DNA according to the following instruction (Suo Laibao, Beijing): each sample was incubated in 100 μL of RNase A at 37° C. for 30 min; 500 μL of PI (propidium iodide) was added to each sample; each sample was incubated at room temperature for 30 min in the dark; the cell cycle was analyzed by a flow cytometry and a ModFit software; and Graphpad prism 6.0 was used to estimate the percentage of a cell population in the different phases of the cell cycle.

FIGS. 3A-3H and 4A-4H show ModFit analysis of the percentage of HepG2 liver cancer cells in different phases affected by the compound ID1120B-1 and its derivatives ID1214B-1, IY1214A-1 and IY1214B-2. FIGS. 5A-5D are a GraphPad Prism analysis of the results in FIGS. 3A-3H and 4A-4H. As shown in FIGS. 3A-3H, 4A-4H and 5A-5D, compared with the negative control (DMSO), the compound ID1120B-1 and its derivatives ID1214B-1, IY1214A-1 and IY1214B-2 dramatically induce the liver cancer cells in the G2/M phase in a dose-dependent manner, and significantly decrease the percentage of the liver cancer cells in the G1/S phase; the compound ID1214B-1 increases the length of the G2 phase from 10.6% to 17.54% of the cell cycle; the compound IY1214A-1 increases the length of the G2 phase from 13.35% to 34.54% of the cell cycle; the compound IY1214B-2 increases the length of the G2 phase from 9.6% to 21.71% of the cell cycle.

### Example 4

Induction of apoptosis in liver cancer cells by compounds IY1214A-1 and IY1214B-2.

The HepG2 cells were harvested during log phase, digested, centrifuged and prepared into a single cell suspension; the number of the cells in the cell suspension was counted; the cells were seeded into a 12-well plate, with 2×10⁵ cells per well; three wells were used as a parallel control design; 16 hours after seeding, the cells were treated with the compounds in a concentration gradient for 48 h; the cells were digested with EDTA-free trypsin and resuspended; the number of the cells in the cell suspension was counted and diluted to 1×10⁶ cells/mL; an annexin V apoptosis detection kit was used according to the following instruction (Suo Laibao, Beijing): the cells were washed twice with 1×PBS (each time the mixture was centrifuged at 6000 rpm for 0.5 min), washed once with 1×Binding buffer (and the mixture was centrifuged at 6000 rpm for 0.5 min); the supernatant was discarded; the cells were resuspended with 300 μL of 1×Binding buffer; 5 μL of Annexin V-FITC was added into each tube, and incubated in the dark for 10 min; 5 μL of PI was added into each tube and incubated in the dark for 5 min; and each tube was then inspected on a machine in the dark.

FIGS. 6A-6B show flow cytometry data of apoptosis in HepG2 liver cancer cells treated with compounds IY1214A-1 and IY1214B-2. The results show that both the compounds IY1214A-1 and IY1214B-2 induce apoptosis in the HepG2 liver cancer cells in a dose-dependent manner compared with the control group. After the cells were treated for 48 h with the compound IY1214A-1 in 4 μM and 8 μM concentrations, the apoptosis rates increase to 57.7% and 63%, respectively, which are more than 2 times higher than that of the three untreated wells; after the cells are treated for 48 h with the compound MIY1214B-2 in a concentration of 8 μL, the apoptosis rate increases to 47.1%, which is 3.3 times higher than that of the three untreated wells.

### Example 5

Regulation of expression of cell cycle regulatory molecules and autophagy-related genes by a compound IY1214B-2.

HepG2 liver cancer cells were seeded in a 6-well plate, with 1×10⁶ cells per well, and treated with the compound IY1214B-2 (in 0 and 10 μM concentrations) for 24 h; total RNA was extracted from the HepG2 liver cancer cells by a single-step TRIzol method; the concentration and purity of the total RNA was measured; the total RNA was used as a template; and complementary DNA (cDNA) was synthesized from the RNA template according to the instruction of a reverse transcription kit (Promega); sqRT-PCR and qPCR were used to quantify the expression of the genes CCNB1, CDK1 and SQSTM; and the gene ACTB was used as an internal reference gene for gene expression normalization. Sequences of primers used to quantify gene expression are listed in Table 2.

TABLE 2
Sequences of primers used to quantify gene
expression

| Gene | Sequence |
|---|---|
| CCNB1-F | TTGGGGACATTGGTAACAAAGTC (SEQ ID NO: 1) |
| CCNB1-R | ATAGGCTCAGGCGAAAGTTTTT (SEQ ID NO: 2) |
| CDK1-F | GGATGTGCTTATGCAGGATTCC (SEQ ID NO: 3) |
| CDK1-R | CATGTACTGACCAGGAGGGATAG (SEQ ID NO: 4) |
| SQSTM1-F | GACTACGACTTGTGTAGCGTC (SEQ ID NO: 5) |
| SQSTM1-R | AGTGTCCGTGTTTCACCTTCC (SEQ ID NO: 6) |
| ATCB-F | CATGTACGTTGCTATCCAGGC (SEQ ID NO: 7) |
| ATCB-R | CTCCTTAATGTCACGCACGAT (SEQ ID NO: 8) |

A 20 μL reaction mix for qPCR contained:

2 μL of cDNA;

10 μL of 2× SYBR Green Supermix;

1 μL of upstream and downstream primers;

0.3 μL of reference dye;

6.7 μL of water.

Each sample has three technical replicates.

Cycling conditions comprised:

pre-denaturation at 95° C. for 5 min;

denaturation at 95° C. for 15 sec;

annealing at 60° C. for 15 sec; and

extension at 72° C. for 30 sec.

After 40 cycles, the cycle threaded (CT) value of the β-actin gene was used as an initial value in comparison with the amount of the amplified product.

FIG. 7 shows qPCR results of regulation of mRNA expression levels of cell cycle regulatory molecules and autophagy-related genes by the compound IY1214B-2. As shown in FIG. 7, when compared with the expression level of the β-actin gene (ATCB), the expressed mRNA levels of two G2 phase regulators Cyclin B1 (gene name: CCNB1) and CDC2 (gene name: CDK1) are down-regulated by 20-25%, and the expression levels of autophagy-related marker p62 (gene name: SQSTM) is up-regulated by 2.5 times. The results show that the compound IY1214B-2 induces cell cycle arrest at the G2/M phase by down-regulating the expression of Cyclin B1 and CDC2 at the level of mRNA regulation, or induces autophagy by up-regulating the expression of P62 at the level of mRNA regulation, thus inhibiting the growth of tumor cells.

The compound ID1120B-1 and its derivatives ID1214B-1, IY1214A-1 and IY1214B-2 are found to inhibit proliferation of cancer cells within liver cancer, breast cancer, lung cancer, gefitinib- or afatinib-resistant lung cancer, or leukemia; specifically; the cancer cells are arrested in G2/M phase of the cell cycle and undergo apoptosis.

The disclosed compounds are suitable for use in treatment of cancers related to abnormal cell proliferation; specifically, the disclosed compounds are altered into pharmaceutically acceptable salts or mixed with drug carriers to form antitumor drugs.

It will be obvious to those skilled in the art that changes and modifications may be made, and therefore, the aim in the appended claims is to cover all such changes and modifications.

What is claimed is:

1. A naphthylurea compound, having the following formula: 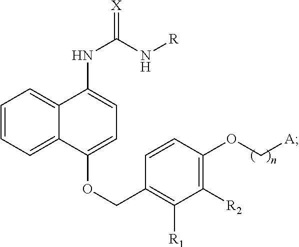
wherein,
R represents H, a C1-C5 straight-chain alkyl, a C1-C5 straight-chain alkyl
with a halogen-substituted end, a 5-8-membered cycloalkyl, 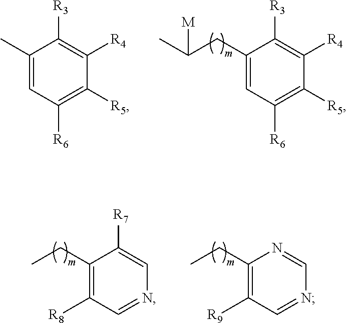
R₁, R₂, R₃, R₄, R₅, R₆, R₇, R₈, R₉ at each occurrence represent H, F, Cl, Br, —CN, —CH₃, —CF₃, —OCH₃, or —OCF₃; R₅ optionally represents phenyl, and M is H or —CH₃;
m represents a number of CH₂, and is 0 or 1;
n represents a number of CH₂, and is 1, 2, 3, or 4;
A is 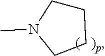
and p represents a number of CH₂, and is 2; and
X is O or S.

2. The compound of claim 1, being one of the following compounds:

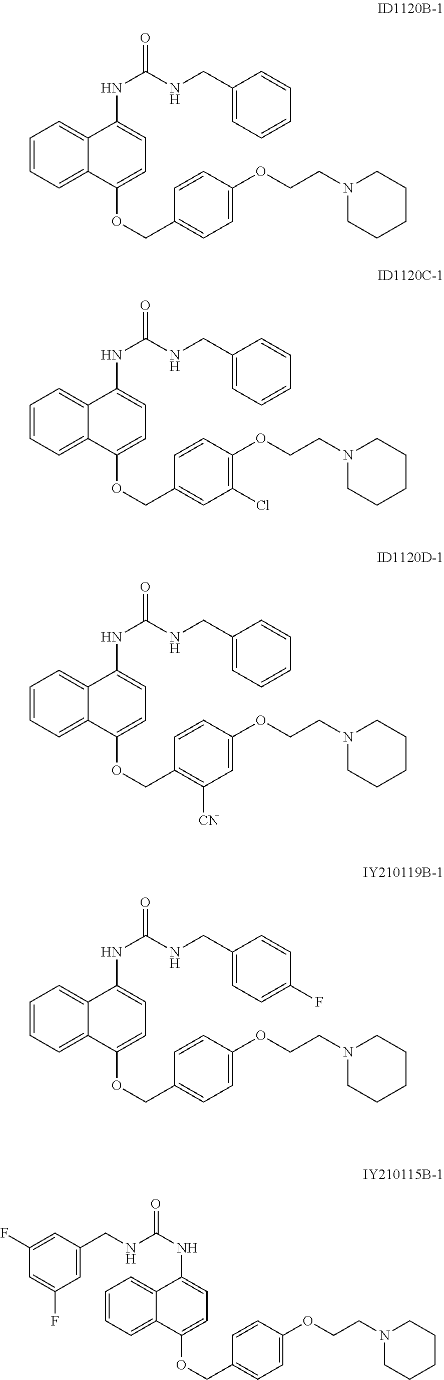 [SMILES: O=C(NCc1ccccc1)Nc1ccc(OCc2ccc(OCCN3CCCCC3)cc2)c2ccccc12 ; O=C(NCc1ccccc1)Nc1ccc(OCc2ccc(OCCN3CCCCC3)c(Cl)c2)c2ccccc12 ; N#Cc1cc(OCCN2CCCCC2)ccc1COc1ccc(NC(=O)NCc2ccccc2)c2ccccc12 ; O=C(NCc1ccc(F)cc1)Nc1ccc(OCc2ccc(OCCN3CCCCC3)cc2)c2ccccc12 ; O=C(NCc1cc(F)cc(F)c1)Nc1ccc(OCc2ccc(OCCN3CCCCC3)cc2)c2ccccc12]

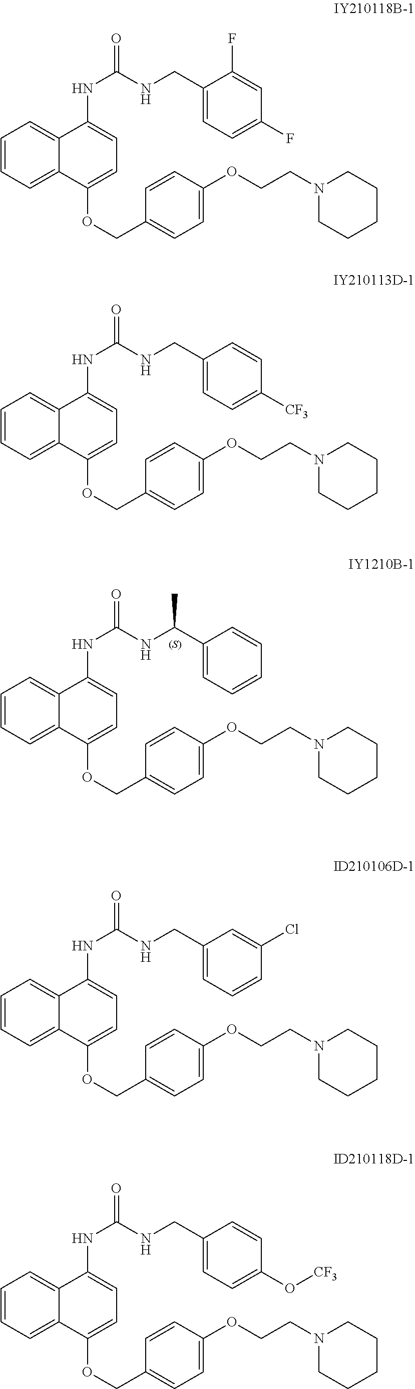

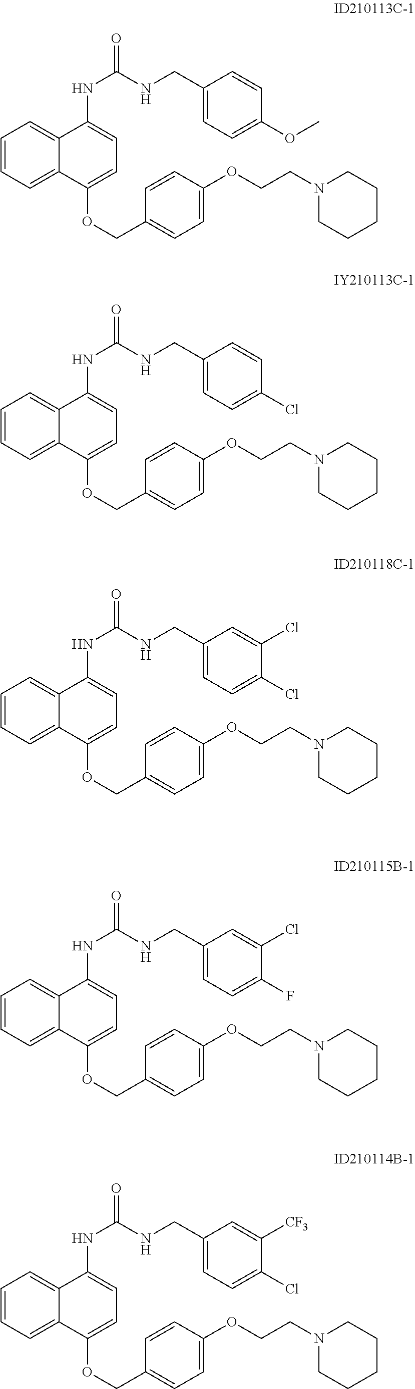 [SMILES: COc1ccc(CNC(=O)Nc2ccc(OCc3ccc(OCCN4CCCCC4)cc3)c3ccccc23)cc1 ; O=C(NCc1ccc(Cl)cc1)Nc1ccc(OCc2ccc(OCCN3CCCCC3)cc2)c2ccccc12 ; O=C(NCc1ccc(Cl)c(Cl)c1)Nc1ccc(OCc2ccc(OCCN3CCCCC3)cc2)c2ccccc12 ; O=C(NCc1ccc(F)c(Cl)c1)Nc1ccc(OCc2ccc(OCCN3CCCCC3)cc2)c2ccccc12 ; O=C(NCc1ccc(Cl)c(C(F)(F)F)c1)Nc1ccc(OCc2ccc(OCCN3CCCCC3)cc2)c2ccccc12]

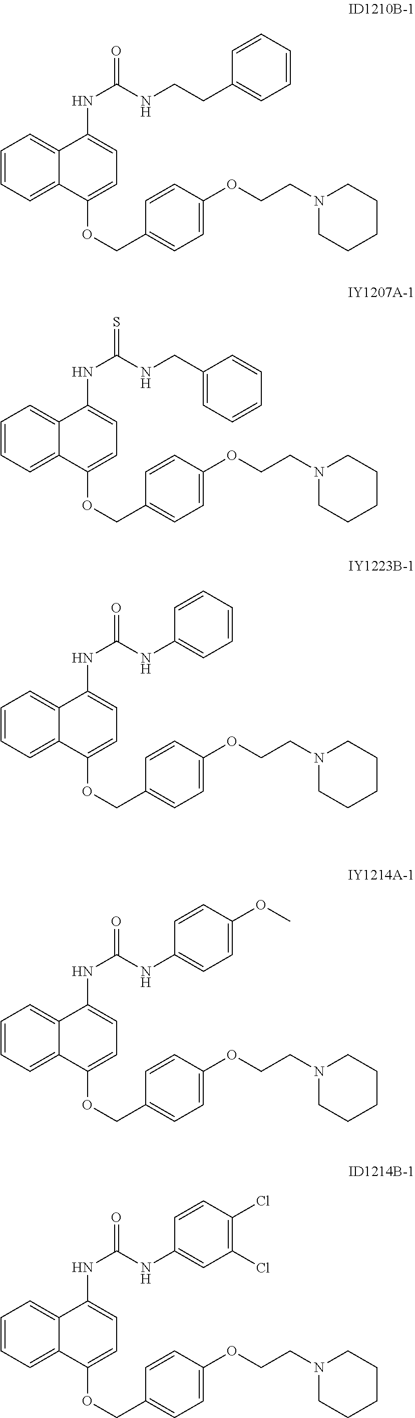 [SMILES: O=C(NCCc1ccccc1)Nc1ccc(OCc2ccc(OCCN3CCCCC3)cc2)c2ccccc12 ; S=C(NCc1ccccc1)Nc1ccc(OCc2ccc(OCCN3CCCCC3)cc2)c2ccccc12 ; O=C(Nc1ccccc1)Nc1ccc(OCc2ccc(OCCN3CCCCC3)cc2)c2ccccc12 ; COc1ccc(NC(=O)Nc2ccc(OCc3ccc(OCCN4CCCCC4)cc3)c3ccccc23)cc1 ; O=C(Nc1ccc(Cl)c(Cl)c1)Nc1ccc(OCc2ccc(OCCN3CCCCC3)cc2)c2ccccc12]

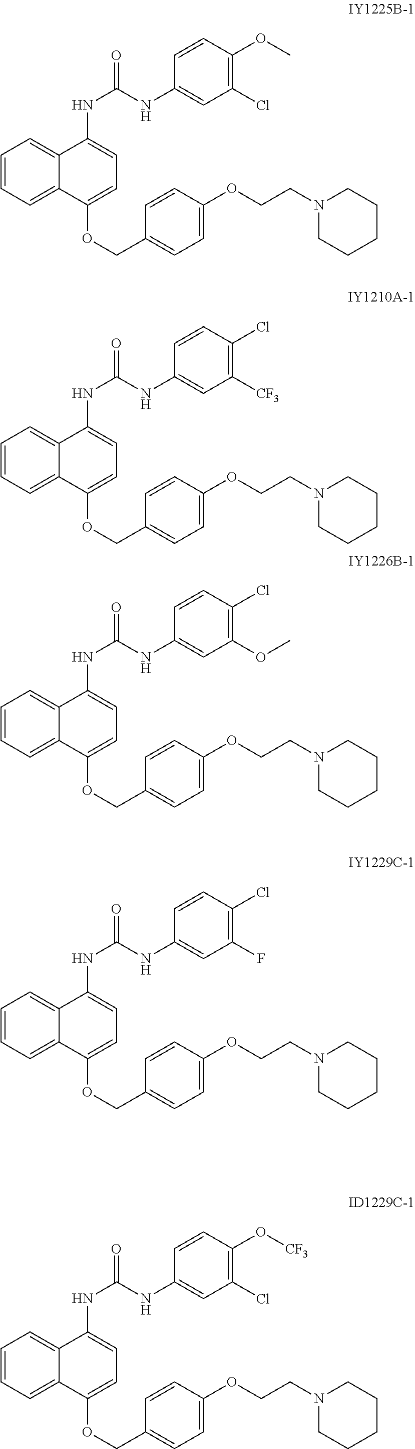 [SMILES: COc1ccc(NC(=O)Nc2ccc(OCc3ccc(OCCN4CCCCC4)cc3)c3ccccc23)cc1Cl ; O=C(Nc1ccc(Cl)c(C(F)(F)F)c1)Nc1ccc(OCc2ccc(OCCN3CCCCC3)cc2)c2ccccc12 ; COc1cc(NC(=O)Nc2ccc(OCc3ccc(OCCN4CCCCC4)cc3)c3ccccc23)ccc1Cl ; O=C(Nc1ccc(Cl)c(F)c1)Nc1ccc(OCc2ccc(OCCN3CCCCC3)cc2)c2ccccc12 ; O=C(Nc1ccc(OC(F)(F)F)c(Cl)c1)Nc1ccc(OCc2ccc(OCCN3CCCCC3)cc2)c2ccccc12]

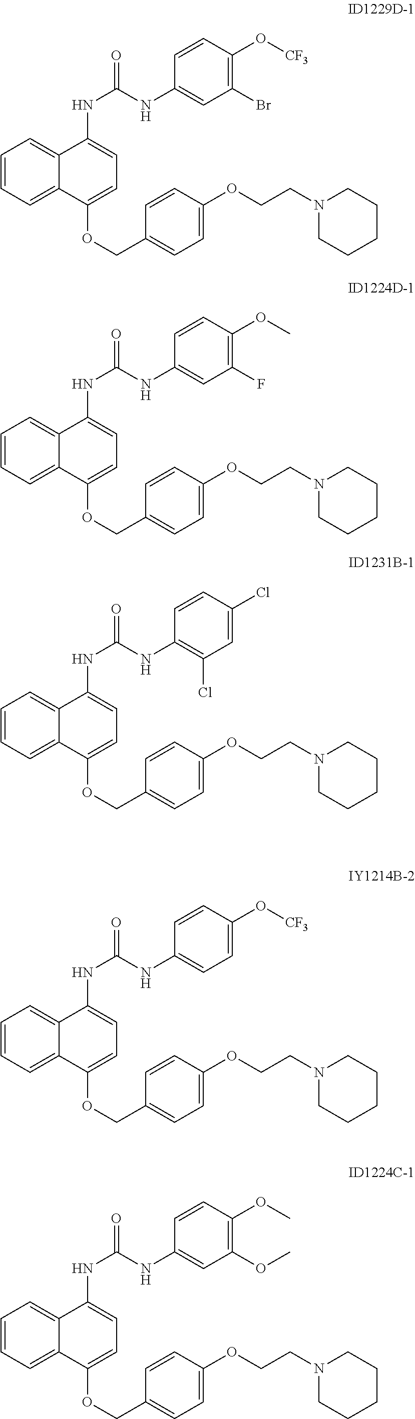 [SMILES: O=C(Nc1ccc(OC(F)(F)F)c(Br)c1)Nc1ccc(OCc2ccc(OCCN3CCCCC3)cc2)c2ccccc12 ; COc1ccc(NC(=O)Nc2ccc(OCc3ccc(OCCN4CCCCC4)cc3)c3ccccc23)cc1F ; O=C(Nc1ccc(Cl)cc1Cl)Nc1ccc(OCc2ccc(OCCN3CCCCC3)cc2)c2ccccc12 ; O=C(Nc1ccc(OC(F)(F)F)cc1)Nc1ccc(OCc2ccc(OCCN3CCCCC3)cc2)c2ccccc12 ; COc1ccc(NC(=O)Nc2ccc(OCc3ccc(OCCN4CCCCC4)cc3)c3ccccc23)cc1OC]

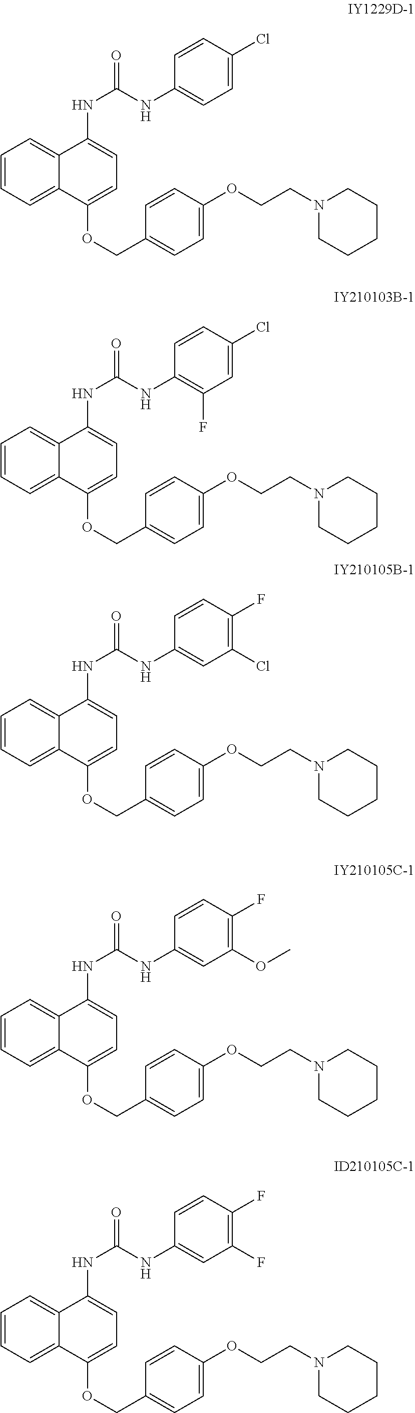 [SMILES: O=C(Nc1ccc(Cl)cc1)Nc1ccc(OCc2ccc(OCCN3CCCCC3)cc2)c2ccccc12 ; O=C(Nc1ccc(Cl)cc1F)Nc1ccc(OCc2ccc(OCCN3CCCCC3)cc2)c2ccccc12 ; O=C(Nc1ccc(F)c(Cl)c1)Nc1ccc(OCc2ccc(OCCN3CCCCC3)cc2)c2ccccc12 ; COc1cc(NC(=O)Nc2ccc(OCc3ccc(OCCN4CCCCC4)cc3)c3ccccc23)ccc1F ; O=C(Nc1ccc(F)c(F)c1)Nc1ccc(OCc2ccc(OCCN3CCCCC3)cc2)c2ccccc12]

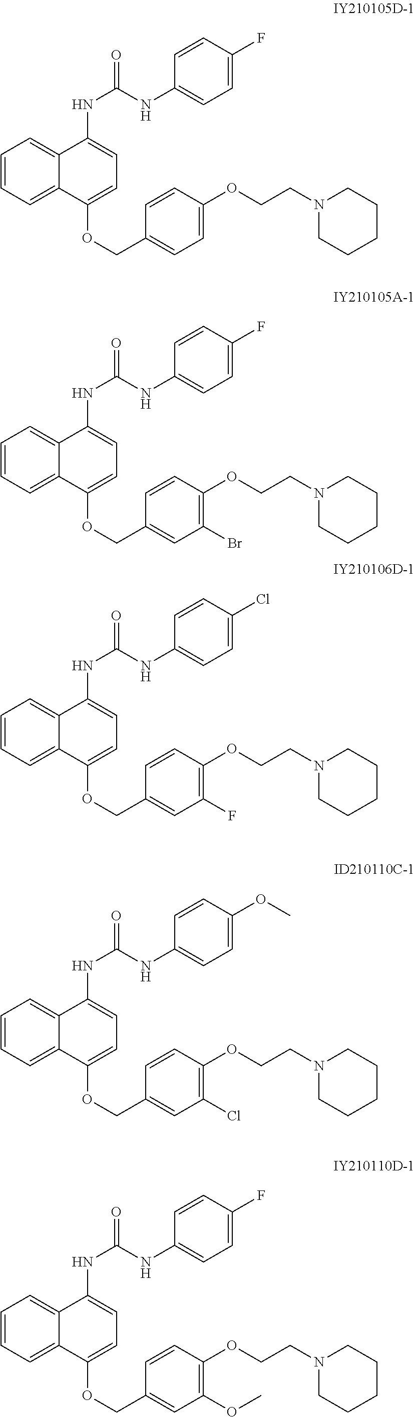 [SMILES: O=C(Nc1ccc(F)cc1)Nc1ccc(OCc2ccc(OCCN3CCCCC3)cc2)c2ccccc12 ; O=C(Nc1ccc(F)cc1)Nc1ccc(OCc2ccc(OCCN3CCCCC3)c(Br)c2)c2ccccc12 ; O=C(Nc1ccc(Cl)cc1)Nc1ccc(OCc2ccc(OCCN3CCCCC3)c(F)c2)c2ccccc12 ; COc1ccc(NC(=O)Nc2ccc(OCc3ccc(OCCN4CCCCC4)c(Cl)c3)c3ccccc23)cc1 ; COc1cc(COc2ccc(NC(=O)Nc3ccc(F)cc3)c3ccccc23)ccc1OCCN1CCCCC1]

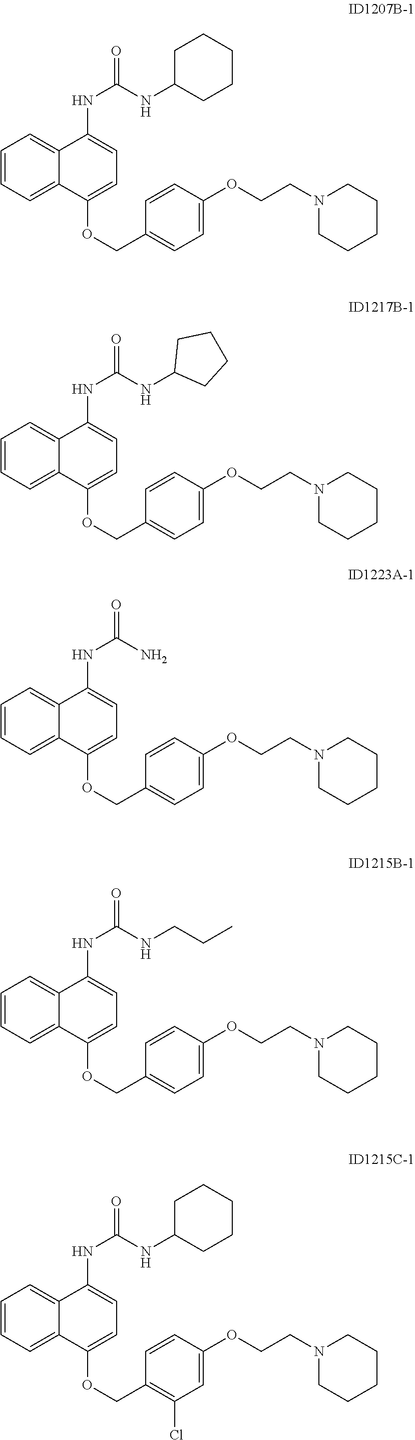 [SMILES: O=C(Nc1ccc(OCc2ccc(OCCN3CCCCC3)cc2)c2ccccc12)NC1CCCCC1 ; O=C(Nc1ccc(OCc2ccc(OCCN3CCCCC3)cc2)c2ccccc12)NC1CCCC1 ; NC(=O)Nc1ccc(OCc2ccc(OCCN3CCCCC3)cc2)c2ccccc12 ; CCCNC(=O)Nc1ccc(OCc2ccc(OCCN3CCCCC3)cc2)c2ccccc12 ; O=C(Nc1ccc(OCc2ccc(OCCN3CCCCC3)cc2Cl)c2ccccc12)NC1CCCCC1]

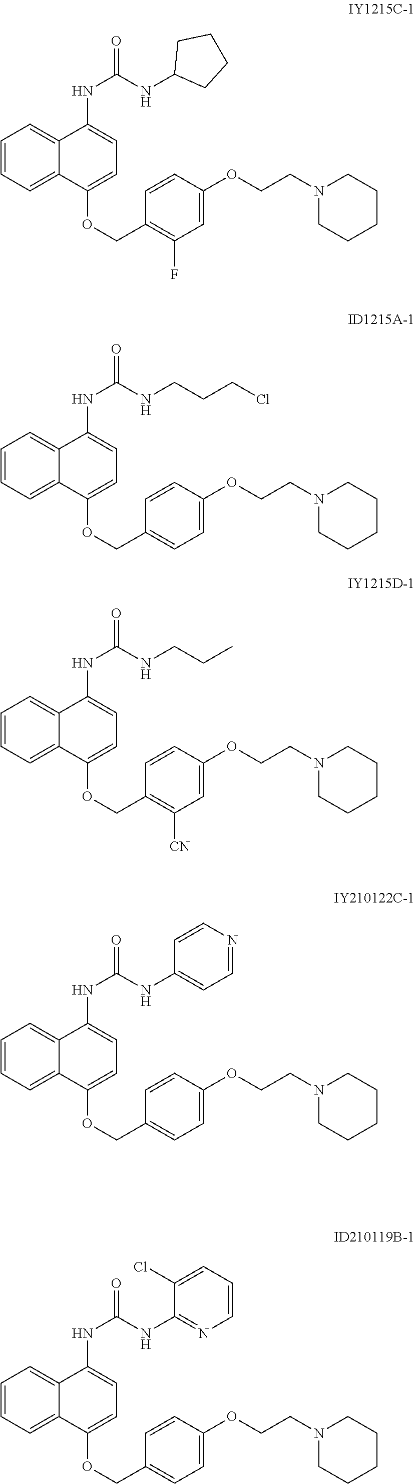 [SMILES: O=C(Nc1ccc(OCc2ccc(OCCN3CCCCC3)cc2F)c2ccccc12)NC1CCCC1 ; O=C(NCCCCl)Nc1ccc(OCc2ccc(OCCN3CCCCC3)cc2)c2ccccc12 ; CCCNC(=O)Nc1ccc(OCc2ccc(OCCN3CCCCC3)cc2C#N)c2ccccc12 ; O=C(Nc1ccncc1)Nc1ccc(OCc2ccc(OCCN3CCCCC3)cc2)c2ccccc12 ; O=C(Nc1ncccc1Cl)Nc1ccc(OCc2ccc(OCCN3CCCCC3)cc2)c2ccccc12]

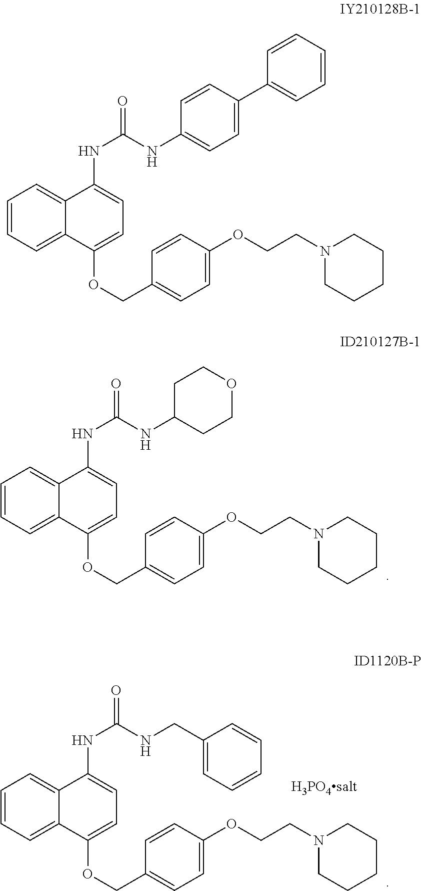 [SMILES: O=C(Nc1ccc(-c2ccccc2)cc1)Nc1ccc(OCc2ccc(OCCN3CCCCC3)cc2)c2ccccc12 ; O=C(Nc1ccc(OCc2ccc(OCCN3CCCCC3)cc2)c2ccccc12)NC1CCOCC1 ; O=C(NCc1ccccc1)Nc1ccc(OCc2ccc(OCCN3CCCCC3)cc2)c2ccccc12]

3. A biologically acceptable salt, being formed by contacting the compound of claim 1 with at least an acid selected from the group consisting of acetic acid, dihydrofolic acid, benzoic acid, citric acid, sorbic acid, propionic acid, oxalic acid, fumaric acid, maleic acid, hydrochloric acid, malic acid, phosphoric acid, sulfite, sulfuric acid, vanillic acid, tartaric acid, ascorbic acid, boric acid, lactic acid, and ethylenediaminetetraacetic acid.

4. A method for preparing the compound of claim 1, comprising:
1) dissolving 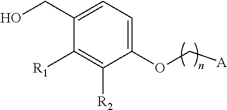
in tetrahydrofuran to yield a mixture, adding NaH in batches at −5-5° C. to the mixture, adding 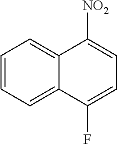 [SMILES: O=[N+]([O-])c1ccc(F)c2ccccc12]
to the mixture, and stirring at room temperature, to yield 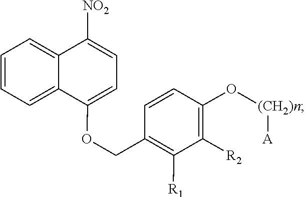
2) dissolving 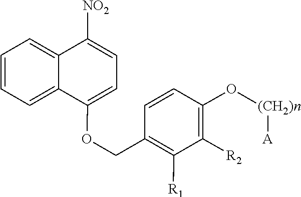
in a mixture solution of ethanol and saturated ammonium chloride aqueous solution, adding iron powders to the mixture solution at 40-50° C. and stirring at 50-60° C., to yield 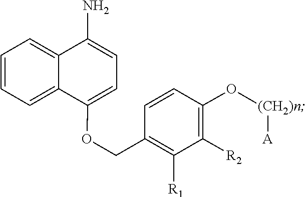
and
3) dissolving 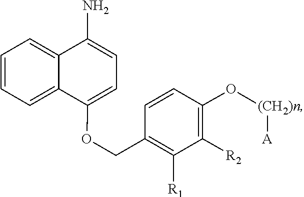
R-isocyanate or R-isothiocyanate, and N,N-diisopropylethylamine in 1,2-dichloroethane, stirring at 80-90° C., and extracting through column chromatography to yield

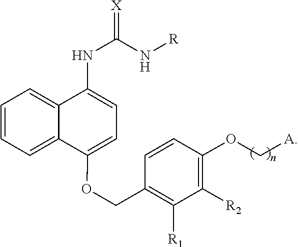

5. The method of claim 4, wherein 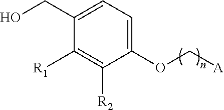
is prepared as follows:
a) dissolving 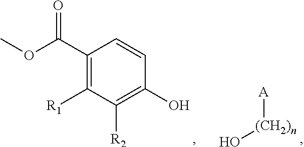
and triphenylphosphine in tetrahydrofuran, and adding diisopropyl azodicarboxylate to a resulting mixture at −5-5° C. under protective atmosphere, and stirring at room temperature, to yield 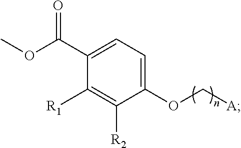
and
b) dissolving 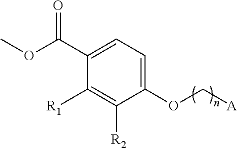
in tetrahydrofuran, adding lithium aluminum hydride in batches at −5-5° C., and stirring at room temperature, to yield

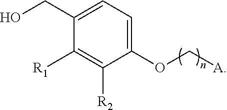

6. The method of claim 4, wherein
in 1), a molar ratio of 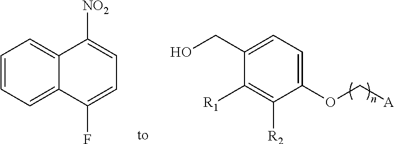
to NaH is 1: 1.2:2;
in 2), a molar ratio of 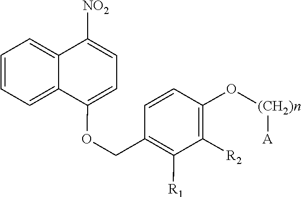
to the iron powders is 1:5, and a volume ratio of ethanol and the saturated ammonium chloride aqueous solution is 1:1; and
in 3), a molar ratio of 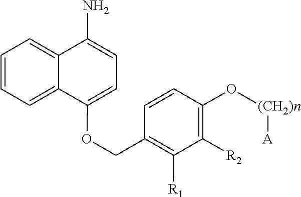
to R-isocyanate or R-isothiocyanate, and to N,N-diisopropylethylamine is 1:1.2:2.0.

7. The method of claim 5, wherein in a), a molar ratio of 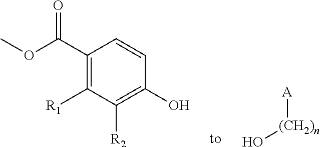
to triphenylphosphine to diisopropyl azodicarboxylate is 1:1.2:1.2: 1.2; and in b), a molar ratio of 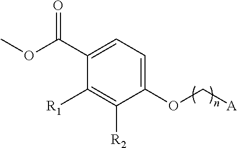
to lithium aluminum hydride is 1:1.

8. A method for treating a tumor comprising administering a patient in need thereof a naphthylurea compound of claim 1 or a biologically acceptable salt thereof.

9. The method of claim 8, wherein the tumor is liver cancer, breast cancer, lung cancer, or leukemia.


SEQUENCE LISTING (8 sequences)

SEQ ID NO: 1 - DNA, 23 bp, synthetic construct
  source 1..23: mol_type = other DNA
ttggggacattggtaacaaagtc

SEQ ID NO: 2 - DNA, 22 bp, synthetic construct
  source 1..22: mol_type = other DNA
ataggctcaggcgaaagttttt

SEQ ID NO: 3 - DNA, 22 bp, synthetic construct
  source 1..22: mol_type = other DNA
ggatgtgcttatgcaggattcc

SEQ ID NO: 4 - DNA, 23 bp, synthetic construct
  source 1..23: mol_type = other DNA
catgtactgaccaggagggatag

SEQ ID NO: 5 - DNA, 21 bp, synthetic construct
  source 1..21: mol_type = other DNA
gactacgacttgtgtagcgtc

SEQ ID NO: 6 - DNA, 21 bp, synthetic construct
  source 1..21: mol_type = other DNA
agtgtccgtgtttcaccttcc

SEQ ID NO: 7 - DNA, 21 bp, synthetic construct
  source 1..21: mol_type = other DNA
catgtacgttgctatccaggc

SEQ ID NO: 8 - DNA, 21 bp, synthetic construct
  source 1..21: mol_type = other DNA
ctccttaatgtcacgcacgat